(19) United States
(12) Patent Application Publication
(10) Pub. No.: US 2007/0092467 A1
(43) Pub. Date: Apr. 26, 2007

(54) Oxyacetamide compounds useful for stimulating or inducing the growth and/or retarding the loss of keratin fibers

(75) Inventors: Roger Rozot, Lagny/Marne (FR); Philippe Breton, Noisy Le Roi (FR); Michel Neuwels, Waterloo (BE); Christophe Boulle, Parris (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/583,792

(22) Filed: Oct. 20, 2006

Related U.S. Application Data
(63) Continuation of application No. PCT/FR05/00926, filed on Apr. 18, 2005.

(30) Foreign Application Priority Data
Apr. 22, 2004 (FR) 04/04298

(51) Int. Cl. A61K 8/49; A61K 31/695; A61K 31/541; A61K 31/5377; A61K 31/496; A61K 31/506

(52) U.S. Cl. 424/70.1; 514/63; 514/210.17; 514/275; 514/227.5; 514/235.5; 514/235.2; 514/252.14; 514/341; 514/340; 514/406

(57) ABSTRACT

Oxyacetamide compounds that are inhibitors of type-1 15-hydroxyprostaglandin dehydrogenase (15-PGDH) and which have the formula (I), or salts and/or solvates thereof: 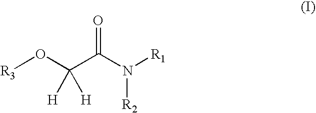 are useful for inducing and/or stimulating the growth of human keratin fibers, e.g., human head hair and/or eyelashes, and/or for retarding the loss and/or increasing the density thereof and/or for caring for and/or making up such keratin fibers.

### CROSS-REFERENCE TO PRIORITY/PCT/PROVISIONAL APPLICATIONS

This application claims priority under 35 U.S.C. § 119 of FR 04/04298, filed Apr. 22, 2004, and of Provisional Application No. 60/566,599, filed Apr. 30, 2004, and is a continuation of PCT/FR 2005/000926 filed Apr. 18, 2005 and designating the United States, published in the English language as WO 2005/107687 A1 on Nov. 17, 2005, each hereby expressly incorporated by reference and each assigned to the assignee hereof.

### BACKGROUND OF THE INVENTION

1. Technical Field Of The Invention

The present invention relates to compositions for caring for or making up keratin fibers, containing an effective amount of a 2-oxyacetamide compound, for increasing the density of these fibers and/or improving their appearance. More especially, this invention relates to compositions for caring for or making up human hair or the eyelashes, for inducing and/or stimulating their growth and/or retarding their loss.

The present invention also relates to novel 2-oxyacetamide compounds and to a cosmetic treatment regime or regimen for stimulating the growth of keratin fibers and in particular the hair, and/or retarding loss thereof.

The human keratin fibers to which this invention relates are especially head hair, the eyebrows, the eyelashes, beard hair, moustache hair and pubic hair. The present invention relates more especially to human head hair and/or eyelashes.

In particular, this invention relates to compositions for topical application for caring for and/or making up the hair or the eyelashes, containing a 2-oxyacetamide compound, for increasing the density and/or improving the appearance thereof.

2. Description of Background and/or Related and/or Prior Art

Hair growth and hair renewal are mainly determined by the activity of the hair follicles and of their matrix environment. Their activity is cyclical and comprises essentially three phases, namely, the anagenic phase, the catagenic phase and the telogenic phase.

The anagenic phase (active phase or growth phase), which lasts several years and during which the hair gets longer, is followed by a very short and transient catagenic phase that lasts a few weeks. During this phase, the hair undergoes a change, the follicle becomes atrophied and its dermal implantation appears higher and higher.

The terminal phase or telogenic phase, which lasts a few months, corresponds to a resting phase of the follicle and the hair ends up by falling out. At the end of this rest period, a new follicle is regenerated in situ and another cycle begins.

The head of hair is thus under permanent renewal, and, out of the approximately 150,000 hairs that make up a head of hair, about 10% are at rest and will be replaced within a few months.

The natural loss or falling-out of the hair may be estimated, on average, as being a few hundred hairs per day for a normal physiological state. This process of permanent physical renewal undergoes a natural change during aging, the hairs become finer and their cycles shorter.

In addition, various causes may result in a substantial, temporary or permanent loss of hair. This may be loss and impairment of hair at the terminal stage of pregnancy (post-partum), during states of dietary malnutrition or imbalance, during physiological stress, or during states of asthenia or of hormonal dysfunction, as may be the case during or at the terminal stage of the menopause. It may also be a case of loss or impairment of the hair related to seasonal phenomena.

It may also be a matter of alopecia, which is essentially due to a disturbance in hair renewal, resulting, in a first stage, in acceleration of the frequency of the cycles to the detriment of the quality of the hair, and then of their quantity. The successive growth cycles result in hairs that are finer and finer and shorter and shorter, gradually transforming into an unpigmented down. Certain areas are preferentially affected, especially the temporal or frontal lobes in men, and a diffuse alopecia of the crown of the head is observed in women.

The term alopecia also covers a whole family of afflictions of hair follicles whose final consequence is the permanent, partial or general loss of the hair. This is more particularly a matter of androgenic alopecia. In a large number of cases, early loss of hair occurs in genetically predisposed individuals; this is then a matter of andro-chrono-genetic alopecia. This form of alopecia especially affects men.

In certain dermatoses of the scalp with an inflammatory component, for instance psoriasis or seborrhoeic dermatitis, hair loss may be greatly accentuated or may result in highly disrupted follicular cycles.

The cosmetics and pharmaceutical industries have for many years been investigating compositions for eliminating or reducing alopecia, and especially for inducing or stimulating hair growth or reducing its loss.

In this perspective, a large number of compositions comprising very diverse active agents have already been developed, for instance 2,4-diamino-6-piperidinopyrimidine 3-oxide, or “minoxidil”, described in U.S. Pat. Nos. 4,139,619 and 4,596,812, or the numerous derivatives thereof such as those described in EP-0,353,123, EP-0,356,271, EP-0,408,442, EP-0,522,964, EP-0,420,707, EP-0,459,890 and EP-0,519,819.

Clinical studies have shown that PGF2-α analogs have the property of inducing the growth of body hairs and eyelashes in man and animals (Murray A. and Johnstone M. D., 1997, Am. J. Opht., 124(4), 544-547). In man, tests conducted on the scalp have indicated that a prostaglandin E2 analog (viprostol) has the property of increasing the hair density (Roenigk H. H., 1988, Clinic Dermatol., 6(4), 119-121).

Moreover, WO 98/33497 describes pharmaceutical compositions containing prostaglandins or prostaglandin derivatives, for combating hair loss in man. Prostaglandins of the type A2, F2α and E2 are mentioned as being preferred.

However, prostaglandins are molecules with a very short biological half-life, which act in an autocrine or paracrine manner, this reflecting the local and labile nature of the metabolism of prostaglandins (Narumiya S. et al., 1999, Physiol. Rev., 79(4), 1193-1226).

It is thus seen to be important, in order to maintain and/or increase hair density in man, to preserve the endogenous reserves of PGF2-α and similarly of PGE2 in various compartments of the hair follicle or its immediate cutaneous environment.

One solution that provides good results is the administration of lipoxygenase-inhibiting compounds and/or cyclooxygenase-inducing compounds to promote hair growth; one theory is that administration of such compounds directs the metabolism of fatty acids toward the endogenous synthesis of prostaglandins in preference to other routes.

However, to further improve these results, it would be desirable to be able to prolong the activity of the prostaglandins involved in growing the hair and keeping it alive.

It is moreover well known that the programs of differentiation of the keratinocytes of the epidermis and of the hair follicle are clearly different. Thus, it is known that the keratins of the hair shaft represent a family (Langbein et al., 2001, J. Biol. Chem., 276: 35123-35132) that is different than the one expressed in the epidermis, that differentiation markers such as the keratins K₁ and K₁₀ are not expressed in the hair follicle and in particular in the outer sheath (Lenoir et al., 1988, Dev. Biol., 130: 610-620), that trichohyalin (O'Guin et al., 1992, J. Invest. Dermatol., 98: 24-32) and keratin K6irs (Porter et al., 2001, Br. J. Dermatol., 145: 558-568) are expressed in the hair follicle, in particular in the inner sheath, but not in the epidermis, and that type-1 cyclooxygenase, although expressed in the epidermis, is not expressed in the keratinocytes of the hair follicle but in the dermal papilla (Michelet. et al., 1997, J. Invest. Dermatol., 108: 205-209).

### SUMMARY OF THE INVENTION

It has now been demonstrated that an enzyme specifically involved in the degradation of these prostaglandins is present in the dermal papilla of the hair, which is a compartment that is a decisive factor in the life of the hair. Specifically, the presence of type-1 15-hydroxyprostaglandin dehydrogenase (abbreviated to 15-PGDH) at this level has now been proven. It has also now been shown that the inhibition of type-1 15-PGDH has a beneficial effect on hair growth.

Consequently, the present invention features hair care or hair treatment compositions containing at least one particular inhibitor of type-1 15-hydroxyprostaglandin dehydrogenase and a physiologically acceptable medium.

### DETAILED DESCRIPTION OF BEST MODE AND SPECIFIC/PREFERRED EMBODIMENTS OF THE INVENTION

Type-1 15-PGDH is a key enzyme in the deactivation of prostaglandins, in particular of PGF2-α and PGE2, which are important mediators of the growth and survival of the hair. It corresponds to the classification EC 1.1.1.141 and is NAD⁺-dependent. It has been isolated from pig kidney; its inhibition has especially been observed with a thyroid hormone, triiodothyronine, at doses very much higher than the physiological doses. Type-2 15-PGDH is itself NADP-dependent.

However, it has never been proposed to administer a type-1 15-PGDH inhibitor to maintain and/or increase the density of human keratin fibers and especially human hair, and/or to reduce the heterogeneity of the diameters of the keratin fibers and especially of human hair. The term “increase the density of keratin fibers”, and especially the density of the hair, means increasing the number or diameter of the keratin fibers and especially of the hair per cm² of skin or scalp.

The present inventors have found that certain 2-oxyacetamide compounds of formula (I), in salified or non-salified form, are, surprisingly, endowed with favorable activity on improving the density of human keratin fibers. It has moreover been found that these compounds are inhibitors of type-1 15-hydroxyprostaglandin dehydrogenase.

The present invention thus features compositions for caring for and/or making up keratin fibers, especially the hair, or mascara compositions for topical application, containing, formulated into a physiologically acceptable medium, an effective amount of at least one 2-oxyacetamide compound of formula (I), or a salt and/or solvate thereof: 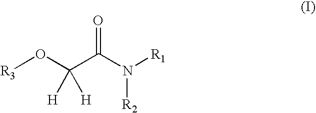
in which:
a) R₁ and R₂ are independently selected from:

1) a hydrogen atom, with R₁ different than R₂,

2) a C₁-C₂₀ alkyl radical optionally substituted with at least one substituent A₁,

3) a hydrocarbon-based ring C¹ of 3 to 7 atoms optionally fused to at least one ring C² of 4 to 7 atoms, the ring C² optionally containing at least one heteroatom to form a heterocycle Hy₂, these rings C¹ and C² optionally comprising a carbonyl or thiocarbonyl function and/or being substituted with at least one substituent A₂,

4) a heterocycle Hy₁ optionally fused to a ring C² of 4 to 7 atoms, the ring C² optionally containing at least one heteroatom to form a heterocycle Hy₂, these rings Hy₁ and C² optionally comprising a carbonyl or thiocarbonyl function and/or being substituted with at least one substituent A₁,

5) one of the groups C(═NR)R′, C(═NR)NR′R″, COR, CSR, COOR, CONRR′, SO₂R or SO₂NRR′;

b) R₃ is selected from:

1) a C₁-C₂₀ alkyl radical optionally substituted with at least one substituent A₁,

2) a hydrocarbon-based ring C³ of 3 to 7 atoms optionally fused to at least one ring C⁴ of 4 to 7 atoms optionally containing at least one heteroatom to form a heterocycle Hy₄, these rings C³ and C⁴ optionally comprising a carbonyl or thiocarbonyl function and/or being substituted with at least one substituent A₂,

3) a heterocycle Hy₃ selected from among pyrrole, furan, thiophene and pyrazole rings and optionally fused to a ring C⁵ constituting a phenyl, a pyridine or a pyrimidine, the heterocycle Hy₃ and the ring C⁵ being optionally substituted with at least one substituent A₁,

4) a heterocycle Hy₅ different than Hy₃ and optionally fused to a ring C⁶ of 4 to 7 atoms, these rings Hy₅ and C⁶ optionally comprising a carbonyl or thiocarbonyl function and/or being substituted with at least one substituent A₁, this ring C⁶ optionally containing at least one heteroatom to form a heterocycle Hy₆;

c) R, R′ and R″ are independently selected from:

1) a hydrogen atom,

2) a C₁-C₂₀ alkyl radical optionally substituted with at least one substituent A₁,

3) a ring C⁷ of 4 to 7 atoms optionally containing at least one heteroatom to form a heterocycle Hy₇ and/or being optionally fused to a ring C⁸ of 4 to 7 atoms optionally containing at least one heteroatom to form a heterocycle Hy₈, the rings C⁷ and C⁸ being optionally substituted with at least one substituent A₁ and optionally comprising a carbonyl or thiocarbonyl function;

d) A₁ is selected from:

1) a halogen atom,

2) a C₁-C₂₀ alkyl radical optionally substituted with a group OR₅,

3) a ring C⁹ of 4 to 15 atoms optionally containing at least one heteroatom to form a heterocycle Hy₉ and/or being optionally fused to a ring C¹⁰ of 4 to 7 atoms, these rings C⁹ and C¹⁰ optionally comprising a carbonyl or thiocarbonyl function and/or at least one substituent A₃ selected from OR₅, CF₃, a halogen and a C₁-C₂₀ alkyl radical,

4) one of the groups CF₃, CN, OR₄, SR₄, NR₄R′₄, NR₄C(═NR′₄)NR″₄R₄′, COR₄, CSR₄, COOR₄, CONR₄R′₄, NR₄COR′₄, NR₄CONR′₄R″₄, SO₂NR₄R′₄, NR₄SO₂R′₄, SO₂R₄, SiR₄R′₄R″₄, Si(OR₄)(OR′₄)OR″₄ and SO₃H, in which R₄, R′₄, R″₄ and R₄′ independently represent a hydrogen atom or a C₁-C₂₀ alkyl radical optionally substituted with a heterocycle Hy₁₀ or a group CONR₅R′₅;

e) A₂ is selected from:

1) a halogen atom,

2) one of the groups CF₃, CN, OR₄, SR₄, NR₄R′₄, OCOR₄, COR₄, CSR₄, COOR₄, SO₂R₄, SiR₄R′₄R″₄, NO₂ and OCF₃, in which R₄, R′₄ and R″₄ independently represent a hydrogen atom or a C₁-C₂₀ alkyl radical optionally substituted with a phenyl radical,

3) a C₁-C₂₀ alkyl radical optionally substituted with a group OR₅,

4) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function and/or at least one substituent A₃ selected from OR₅, CF₃, a halogen atom and a C₁-C₂₀ alkyl radical;

f) R₅ and R′₅ are independently selected from:

1) a hydrogen atom,

2) a C₁-C₂₀ alkyl radical;

g) Hy₁, Hy₂, Hy₄ to Hy₈, Hy₁₀ and Hy₁₁ independently represent a heterocycle of 4 to 7 atoms optionally having from 1 to 4 heteroatoms selected from among N, O and S and combinations thereof and/or comprising a carbonyl or thiocarbonyl function,

h) Hy₉ is a heterocycle of 4 to 15 atoms optionally having from 1 to 5 heteroatoms selected from among N, O and S and combinations thereof and/or comprising a carbonyl or thiocarbonyl function.

The invention also relates to the administration, especially the cosmetic application, of at least one 2-oxyacetamide compound of formula (I) or a salt and/or solvate thereof, as defined above, as an active agent for inducing and/or stimulating the growth of keratin fibers, in particular human keratin fibers, and/or for retarding the loss and/or increasing the density thereof.

This invention also features the cosmetic administration of at least one 2-oxyacetamide compound of formula (I) or a salt and/or solvate thereof, in a composition for caring for or treating keratin fibers, especially human keratin fibers, for retarding the loss and/or increasing the density thereof. This composition also makes it possible to maintain the keratin fibers in good condition and/or to improve their appearance.

The present invention also features the use of at least one 2-oxyacetamide compound of formula (I) or a salt and/or solvate thereof for the formulation of compositions for caring for or treating keratin fibers, especially human keratin fibers, for inducing and/or stimulating the growth of said fibers and/or retarding the loss and/or increasing the density thereof.

This invention also applies to the keratin fibers of mammalian animals (for example dogs, horses or cats).

The human keratin fibers to which the present invention relates are especially head hair, the eyebrows, the eyelashes, beard hair, moustache hair and pubic hair. This invention relates more especially to human head hair and/or eyelashes.

Thus, the present invention also features the cosmetic administration of at least one 2-oxyacetamide compound of formula (I) or a salt and/or solvate thereof, formulated into a human cosmetic hair care composition, for retarding the loss and/or increasing the density of the hair and/or for treating andro-chrono-genetic alopecia. This invention also features the use of at least one 2-oxyacetamide compound of formula (I) or a salt and/or solvate thereof, for the preparation of a human hair composition for inducing and/or stimulating the growth and/or retarding the loss and/or increasing the density of the hair and/or for treating androgenic alopecia.

This invention also features the cosmetic administration of at least one 2-oxyacetamide compound of formula (I) or a salt and/or solvate thereof, in a human cosmetic hair care composition, for treating alopecia of natural origin and in particular androgenic alopecia, and also to the use of at least one 2-oxyacetamide compound of formula (I) or a salt and/or solvate thereof, for the formulation of human hair care compositions for treating alopecia of natural origin and in particular androgenic alopecia. Thus, these compositions make it possible to maintain the head of hair in good condition and/or to combat natural hair loss, more especially in men.

The present invention also features the cosmetic administration of at least one 2-oxyacetamide compound of formula (I) or a salt and/or solvate thereof, in a cosmetic composition for caring for and/or making up human eyelashes, for inducing and/or stimulating the growth and/or increasing the density of the eyelashes, and also to the use of at least one 2-oxyacetamide compound of formula (I) or a salt and/or solvate thereof, for the formulation of a composition for caring for or treating human eyelashes, for inducing and/or stimulating the growth and/or increasing the density of the eyelashes. Such compositions thus make it possible to maintain the eyelashes in good condition and/or to improve their condition and/or appearance.

The present invention also features the administration of at least one 2-oxyacetamide compound of formula (I) or a salt and/or solvate thereof, as an inhibitor of type-1 15-hydroxyprostaglandin dehydrogenase, especially of human skin. This invention also features the use of at least one 2-oxyacetamide compound of formula (I) or a salt and/or solvate thereof, for the formulation of compositions for treating disorders associated with the presence of type-1 15-hydroxyprostaglandin dehydrogenase, in particular in man.

The present invention also features a cosmetic regime or regimen for treating human keratin fibers (especially the hair or the eyelashes) and/or the skin from which said fibers emerge, including the scalp and the eyelids, comprising topically applying onto said keratin fibers and/or said skin a cosmetic composition comprising at least one compound of formula (I) or a salt and/or solvate thereof, maintaining the composition in contact with the keratin fibers and/or the skin from which said fibers emerge, and optionally rinsing said fibers and/or said skin.

This treatment regime or regimen does indeed have the characteristics of a cosmetic process insofar as it makes it possible to improve the esthetic appearance of human keratin fibers and especially the hair and the eyelashes by giving them greater vigor and an improved appearance. In addition, it may be carried out daily for several months, without medical prescription.

More especially, the present invention features a cosmetic regime or regimen for caring for human hair and/or the scalp, for improving their condition and/or appearance, comprising topically applying onto the hair and/or the scalp a cosmetic composition comprising an effective amount of at least one compound of formula (I) or a salt and/or solvate thereof, maintaining the composition in contact with the hair and/or the scalp, and optionally rinsing the hair and/or the scalp.

This invention also features a cosmetic regime or regimen for caring for and/or making up human eyelashes, for improving their condition and/or appearance, comprising topically applying onto the eyelashes and/or the eyelids a mascara composition comprising at least one compound of formula (I) or a salt and/or solvate thereof, and leaving it in contact with the eyelashes and/or the eyelids. This mascara composition may be applied alone or as a base coat for a standard pigmented mascara and may be removed like a standard pigmented mascara.

The present invention also features care and/or mascara compositions for the hair or the eyelashes, containing a cosmetically acceptable medium for topical application and a compound of formula (I) or a salt and/or solvate thereof.

This invention also features compositions for caring for and/or making up keratin fibers, comprising, in a physiologically acceptable medium, in particular a cosmetic medium, at least one compound of formula (I) or a salt and/or solvate thereof and at least one additional active agent that promotes the regrowth and/or limits the loss of human keratin fibers, selected from among aminexil, FP receptor agonists, prostaglandins and derivatives thereof, and vasodilators and more especially selected from aminexil, minoxidil, latanoprost, (5E)-7-{(1R,2R,3R,5S)-3,5-dihydroxy-2-[(3R)-3-hydroxy-5-phenylpentyl]cyclopentyl}hept-5-enoic acid, butaprost and travoprost.

The present invention also features the cosmetic administration of a 2-oxyacetamide compound of formula (I) or a salt and/or solvate thereof, as an active agent for preserving the amount and/or the activity of the prostaglandins in the hair follicles.

This invention also features the use of at least one 2-oxyacetamide compound of formula (I) or a salt and/or solvate thereof, for the formulation of compositions for preserving the amount and/or the activity of the prostaglandins in the hair follicles.

The present invention also features novel 2-oxyacetamide compounds that satisfy at least one of the following formulae (II), (III), (IV) and (V), or a corresponding salt and/or solvate thereof:

Formula (II):

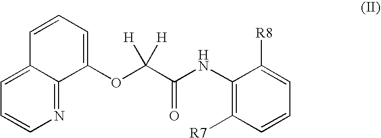

Formula (III):

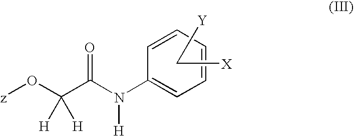

Formula (IV):

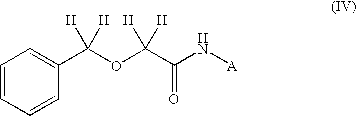

Formula (V): 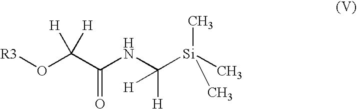
in which:
(i) R7 and R8 independently represent:

1) a hydrogen atom,

2) a halogen F or Cl,

3) one of the groups CF₃, CN, OR₄, SR₄, NHR₆, NR₆R′₆, OCOR₄, COR₄, CSR₄, COOR₄, SO₂R₄, SiR₄R′₄R″₄ and OCF₃, in which R₄, R′₄ and R″₄ independently represent a hydrogen atom or a linear or branched and especially saturated C₁-C₂₀ alkyl radical optionally substituted with a phenyl radical and in which R₆ and R′₆ represent a C₁-C₂₀ alkyl radical optionally substituted with a phenyl radical,

4) a C₁-C₂₀ alkyl radical optionally substituted with a group OR₅ in which R₅ is a hydrogen atom or a linear or branched and especially saturated C₁-C₂₀ alkyl radical,

5) a ring C¹¹ of 4 to 7 atoms optionally containing at least one heteroatom to form a heterocycle Hy₁₁ and/or being optionally fused to a saturated or unsaturated ring C¹² of 4 to 7 atoms, these rings C¹¹ and C¹² optionally comprising a carbonyl or thiocarbonyl function;
(ii) Z is one of the following rings and heterocycles: 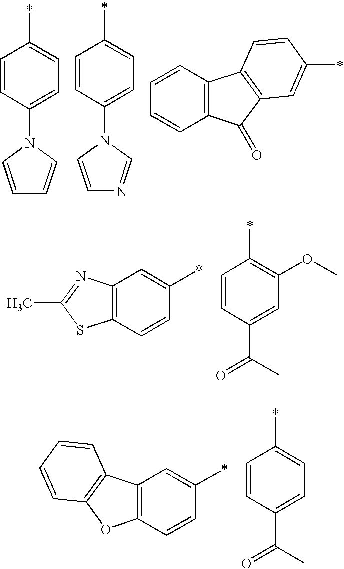
(iii) X and Y independently represent a hydrogen atom or A₂ as defined above (cf. formula (I)) or may form a fused, saturated or unsaturated ring C¹³ of 4 to 7 atoms, this ring C¹³ optionally comprising at least one heteroatom, and being optionally substituted with at least one substituent A₂ and optionally fused to another saturated or unsaturated ring C¹⁴; with the proviso that when Z is the dibenzofuran heterocycle, X and Y independently represent:

1) a hydrogen atom,

2) a halogen atom, for instance F or Cl,

3) one of the groups CF₃, CN, OR₉, SR₄, NR₄R′₄, OCOR₄, COR₄, CSR₄, COOR₄, SO₂R₄, SiR₄R′₄R″₄, NO₂ and OCF₃, in which R₄, R′₄ and R″₄ independently represent a hydrogen atom or a linear or branched and especially saturated C₁-C₂₀ alkyl radical optionally substituted with a phenyl radical and in which R₉ is a hydrogen atom or a linear or branched and especially saturated C₂-C₂₀ alkyl radical optionally substituted with a phenyl radical,

4) a linear or branched and especially saturated C₂-C₂₀ alkyl radical optionally substituted with a group OR₅, 5) a ring C¹¹ of 4 to 7 atoms optionally containing at least one heteroatom to form a heterocycle Hy₁₁, and/or being optionally fused to a ring C¹² of 4 to 7 atoms, these rings C¹¹ and C¹² optionally comprising a carbonyl or thiocarbonyl function; or with the proviso that when Z is the ring: 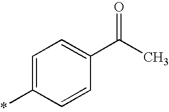
X and Y independently represent:

1) a hydrogen atom, in this case X is different than Y,

2) fluorine,

3) one of the groups OR₁₀, SR₄, NR₄R′₄, OCOR₄, COR₄, CSR₄, COOR₆, SiR₄R′₄R″₄ and OCF₃, in which R₄, R′₄ and R″₄ independently represent a hydrogen atom or a linear or branched and especially saturated C₁-C₂₀ alkyl radical optionally substituted with a phenyl radical, in which R₆ is a linear or branched and especially saturated C₁-C₂₀ alkyl radical optionally substituted with a phenyl radical and in which R₁₀ is a hydrogen atom or a benzyl radical or a linear or branched and especially saturated C₃-C₂₀ alkyl radical optionally substituted with a phenyl radical,

4) a linear or branched and especially saturated C₂-C₂₀ alkyl radical optionally substituted with a group OR₅,

5) X and Y may also form a saturated or unsaturated 5-atom ring C¹³ optionally containing one or two oxygen atoms and being optionally fused to another saturated or unsaturated ring C¹⁴;
(iv) A is one of the following three groups: 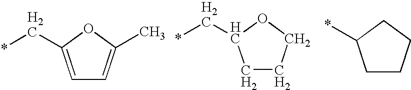
(v) R₃ is as defined above for formula (I).

Advantageously, the compounds of formulae (II), (III), (IV) and (V) are inhibitors of type-1 15-PGDH and preferably selective inhibitors.

The term “15-hydroxyprostaglandin dehydrogenase inhibitor” means a compound of formula (I), which is capable of inhibiting or reducing the activity of the enzyme type-1 15-PGDH, especially the human form thereof, and/or which is capable of inhibiting, reducing or slowing down the reaction catalyzed by this enzyme.

According to one advantageous embodiment of the invention, the compound of formula (I) is a specific inhibitor of 15-PGDH; the term “specific inhibitor” means an active agent that has little or no inhibitory activity on the synthesis of prostaglandins, in particular on the synthesis of PGF2-α or PGE2. According to one particular embodiment of the invention, the 15-PGDH inhibitor has little or no inhibitory activity on the synthesis of prostaglandins, especially on the synthesis of PGF2-α or PGE2. In particular, the type-1 15-PGDH inhibitor has little or no inhibitory activity on prostaglandin synthase (abbreviated as PGF synthase or PGFS).

Specifically, the present inventors have now found that PGF synthase is also expressed in the dermal papilla. Maintaining an effective amount of prostaglandins at the site of action thus results from a complex biological equilibrium from the synthesis and the degradation of these molecules. The exogenous supply of compounds that inhibit the catabolism will thus be less effective if this activity is combined with an inhibition of the synthesis of these prostaglandins.

Advantageously, the compounds of formula (I), in salified or non-salified, and solvated or non-solvated form, have inhibitory activity on 15-PGDH that is higher than the inhibitory activity on PGF synthase. These compounds are referred to as selective inhibitors of 15-PGDH relative to PGF synthase. In particular, the ratio from the inhibitory activities of PGF synthase and of 15-PGDH, respectively, for a given concentration, determined especially by means of the concentration that inhibits 50% of the enzymatic activity of PGF synthase (IC_{50fs}) relative to the concentration that inhibits 50% of the enzymatic activity of 15-PGDH (IC_{50dh}), is at least greater than 1, especially at least 3:1 and advantageously greater than or equal to 5:1.

In the text hereinbelow, unless specifically indicated, the use of the term “compound of formula (I)” should be understood as meaning not only the compound of formula (I) but also one of its salts or solvates, in particular hydrates, or one of its solvated salts (in particular hydrated salts).

According to the invention, the term “salts of a compound of formula (I)” means the organic or mineral salts of a compound of formula (I).

As mineral salts that may be used according to the invention, exemplary are the sodium or potassium salts and also of the ammonium, zinc (Zn²⁺), calcium (Ca²⁺), copper (Cu²⁺), iron (Fe²⁺ and Fe³⁺), strontium (Sr²⁺), magnesium (Mg²⁺) and manganese (Mn²⁺) salts; hydroxides, hydrohalides (for example hydrochlorides), carbonates, hydrogen carbonates, sulfates, hydrogen phosphates and phosphates.

The organic salts according to the invention are, for example, the triethanolamine, monoethanolamine, diethanolamine, hexadecylamine and N,N,N′,N′-tetrakis(2-hydroxypropyl)ethylenediamine salts, and also those of organic acids, for instance citrates, lactates, glycolates, gluconates, acetates, propionates, fumarates, oxalates and tartrates.

As exemplary solvates of the compounds of formula (I), representative are the hydrates, alkoxides or hydroalkoxides.

According to the invention, the compounds of formula (I) are in isolated, i.e., non-polymeric, form.

According to the invention, the term “at least one” means one or more (2, 3 or more). In particular, the composition may contain one or more compounds of formula (I). This or these compound(s) may be cis or trans or Z or E isomers or a mixture of cis/trans or Z/E isomers. They may also be in tautomeric form. This or these compound(s) may be enantiomers and/or diastereoisomers or a mixture of these isomers, in particular a racemic mixture.

For the purposes of the invention, the term “hydrocarbon-based” refers to a group of hydrogen and carbon atoms.

For the purposes of the invention, the term “alkyl radical” means a hydrocarbon-based radical, which may be linear or branched and saturated or unsaturated. In particular, the alkyl radical contains from 1 to 20 and preferably from 1 to 10 carbon atoms. As examples of alkyl radicals that may be used in the invention, representative are methyl, ethyl, isopropyl, n-butyl, tert-butyl, n-hexyl, 2-ethylhexyl, ethylene and propylene radicals.

As halogen atoms according to the invention, representative are chlorine, fluorine and bromine atoms, and preferably chlorine and fluorine atoms.

The rings C¹ to C⁴ and C⁶ to C¹⁴ and also the heterocycles Hy₁, Hy₂ and Hy₄ to Hy₁₁ may be saturated or unsaturated. They in particular comprise 5 to 6 atoms. According to one particular embodiment of the invention, the ring C⁹ may comprise up to 15 atoms and may represent, for example, a crown ether containing 5-CH₂CH₂O— units. In addition, the rings C¹, C³, C⁷, C⁹, C¹¹, C¹³, Hy₁, Hy₃, Hy₅, Hy₇, Hy₉ and Hy₁₁ may be fused to one or more other rings of identical or different chemical nature.

As saturated hydrocarbon-based rings according to the invention, exemplary are cyclopropyl, cyclobutyl, cyclopentyl, cyclohexyl and cycloheptyl radicals. Unsaturated hydrocarbon-based rings that are exemplary include cyclohexenyl and phenyl radicals. As fused hydrocarbon-based rings according to the invention, exemplary are naphthyl and azulenyl radicals. As fused rings of different nature according to the invention, exemplary are benzofuran, dibenzofuran, benzothiophene, benzothiazole, indole, benzimidazole, quinoline, isoquinoline, quinazoline, carboline, chromene, carbazole and fluorene radicals.

According to the invention, R₁ and/or R₂ and/or R₃ may represent a hydrocarbon-based ring as defined above, but also a heterocycle comprising from 1 to 4 heteroatoms selected from among N, O and S, and combinations thereof. In addition, these hydrocarbon-based or heterocyclic rings optionally comprise a carbonyl or thiocarbonyl function.

As examples of rings containing a carbonyl or thiocarbonyl function according to the invention, representative are the following rings:

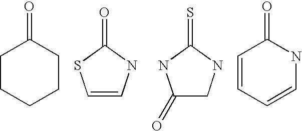

According to one particular embodiment of the invention, R₁ and/or R₂ is a heterocycle Hy₁ selected especially from: azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, dihydrothiazole, thiazolidine, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine, diazepine. In particular, Hy₁, is a γ-butyrolactone, a piperidine or a 1,3-benzodioxole optionally substituted with a linear or branched, and especially saturated, C₁-C₄ alkyl radical.

Advantageously, R₁ is hydrogen and R₂ is one of the following groups:

a linear or branched saturated C₁-C₁₀ alkyl radical, for instance a methyl, ethyl, butyl, propyl, pentyl or hexyl radical, optionally substituted with one or two substituents A₁,

a saturated hydrocarbon-based ring of 3 to 6 carbon atoms,

a saturated or unsaturated heterocycle Hy₁, of 5, 6 or 7 atoms, comprising 1 or 2 heteroatoms selected from among O, N and S and optionally a carbonyl function and/or from 1 to 4 substituents A′₂,

a phenyl ring optionally substituted with one or two substituents A″₂ selected from a halogen (especially fluorine or chlorine), NO₂, OCF₃, CF₃, OR₄, OCH₂R₄, COOR₄, a C₁-C₁₀ alkyl radical that is especially saturated (for instance ethyl, propyl, tert-butyl, hexyl or methyl), and a hydrocarbon-based or heterocyclic aromatic ring,

a phenyl ring fused to one or two saturated or unsaturated hydrocarbon-based or heterocyclic rings of 5 to 6 atoms, thus forming fused rings, for instance indane, carbazole, benzodioxole, fluorene or dibenzofuran rings.

According to the invention, the substituents A₁ or A₂ borne by the same ring or the same alkyl radical may be identical or different. In addition, they may be identical or different for R₁, R₂ and R₃. Similarly, the radicals R₄, R′₄, R″₄ and R₄′ may be identical or different for the same group, the same radical or from a substituent A₁ to a substituent A₂.

In particular, A₁ is a hydrocarbon-based ring or a heterocycle comprising 1 or 2 heteroatoms selected from O and N, this hydrocarbon-based or heterocyclic ring comprising 5 to 6 atoms and optionally a carbonyl function and/or a substituent A₃; or a group selected from SiR₄R′₄R″₄, COOR₄, NR₄R′₄, OR₄, SR₄ and CONR₄R′₄.

According to one particular embodiment of the invention, R₄, R′₄, R″₄ and R₄′ represent a hydrogen atom, a phenyl radical or a C₁-C₁₀ alkyl radical, for instance a methyl, ethyl, tert-butyl, n-butyl, n-propyl, isopropyl or pentyl radical.

Advantageously, A′₂ is a linear or branched and especially saturated C₁-C₁₀ alkyl radical such as methyl.

According to one particular embodiment of the invention, A₃ is a linear or branched and especially saturated C₁-C₁₀ alkyl radical such as methyl, CF₃, a halogen atom, for instance F, OH or OCH₃.

According to one particular embodiment of the invention, R₃ is a heterocycle Hy₅ selected from: azetidine, dihydropyrrole, pyrrolidine, dihydrofuran, tetrahydrofuran, dihydrothiophene, tetrahydrothiophene, imidazole, dihydroimidazole, imidazolidine, thiazole, dihydrothiazole, thiazolidine, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine.

Advantageously, R₃ is one of the following groups:

a saturated or unsaturated hydrocarbon-based ring, optionally substituted with one or more substituents A₂′ selected from a phenyl radical, COOR₄, OR₄, COR₄, CN, a saturated or unsaturated heterocycle containing 5 or 6 atoms and comprising 1 or 2 heteroatoms selected from among O, S and N, for instance pyrrole or imidazole, a halogen (fluorine or chlorine), a phenyl radical optionally substituted with CN, a linear or branched and especially saturated C₁-C₁₀ alkyl radical, for instance ethyl, methyl, isopropyl, isobutyl or tert-butyl, with R₄ representing a hydrogen atom or a linear or branched and especially saturated C₁-C₁₀ alkyl radical, for instance methyl or ethyl,

a phenyl ring fused to one or two saturated or unsaturated hydrocarbon-based or heterocyclic rings C⁴ of 5 to 6 atoms, the ring(s) C⁴ optionally comprising a carbonyl function and/or optionally being substituted with a linear or branched and especially saturated C₁-C₁₀ alkyl radical, for instance methyl or ethyl, this phenyl ring fused to this or these ring(s) C⁴ thus forming fused rings such as indane, benzothiazole, quinoline, carbazole, fluorene, fluorenone or dibenzofuran rings,

a linear or branched and especially saturated C₁-C₁₀ alkyl radical, for instance methyl, optionally substituted with one or two substituents A₁, for instance a phenyl radical.

As examples of heterocycles Hy₂, Hy₄, Hy₆, Hy₇, Hy₈, Hy₉, Hy₁₀ and Hy₁₁ according to the invention, independently representative are azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, thiazole, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine rings or a 15-C-5 crown ether for Hy₉. Preferably, pyrrole, pyrrolidine, imidazole, furan, thiophene, oxazole, thiazole, isoxazole, isothiazole, oxadiazole, pyrazole, tetrazole, pyridine, pyrimidine, triazole, pyrazine, pyridazine, piperidine, piperazine or morpholine rings or a 15-C-5 crown ether for Hy₉ are employed.

According to one preferred embodiment of the invention, the 2-oxyacetamide compounds satisfy formula (1a) below, or a salt and/or a solvate thereof: 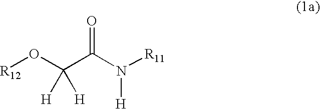
in which:
α) R₁₁ is selected from:

1) a linear or branched and especially saturated C₁-C₁₀ alkyl radical optionally substituted with at least one substituent A₄,

2) a saturated or unsaturated hydrocarbon-based ring C¹⁵ of 5 to 6 atoms optionally substituted with at least one substituent A₅;

β) R₁₂ is selected from:

1) a linear or branched and especially saturated C₁-C₁₀ alkyl radical optionally substituted with at least one substituent A₄,

2) a saturated or unsaturated hydrocarbon-based ring C¹⁶ of 5 to 6 atoms, optionally fused to at least one saturated or unsaturated ring C¹⁷ of 5 to 6 atoms optionally containing at least one heteroatom to form a heterocycle Hy₁₇, these rings C¹⁶ and C¹⁷ optionally comprising a carbonyl function and/or being substituted with at least one substituent A₅;

γ) A₄ is selected from:

1) a saturated or unsaturated ring C¹⁸ of 5 to 6 atoms optionally containing at least one heteroatom to form a heterocycle Hy₁₈, this ring or this heterocycle being optionally substituted with at least one substituent A₆ selected from OR₁₄, CF₃, a halogen atom, for instance fluorine or chlorine, and a linear or branched and especially saturated C₁-C₁₀ alkyl radical;

2) one of the groups CF₃, CN, OR₁₃, SR₁₃, NR₁₃R′₁₃, NR₁₃C(═NR′₁₃)NR″₁₃R′₁₃, COR₁₃, COOR₁₃, CONR₁₃R′₁₃, NR₁₃COR′₁₃, NR₁₃CONR′₁₃R″₁₃, SO₂NR₁₃R′₁₃, NR₁₃SO₂R′₁₃, SO₂R₁₃, SiR₁₃R′₁₃R″₁₃ and SO₃H, in which R₁₃, R′₁₃, R″₁₃ and R₁₃′ independently represent a hydrogen atom or a linear or branched and especially saturated C₁-C₁₀ alkyl radical;

δ) A₅ is selected from:

1) a halogen atom, for instance fluorine or chlorine,

2) one of the groups CF₃, CN, OR₁₅, SR₁₅, NR₁₅R′₁₅, OCOR₁₅, COR₁₅, COOR₁₅, SO₂R₁₅, SiR₁₅R′₁₅R″₁₅, NO₂ and OCF₃, in which R₁₅, R′₁₅ and R″₁₅ independently represent a hydrogen atom or a linear or branched and especially saturated C₁-C₁₀ alkyl radical optionally substituted with a phenyl radical,

3) a linear or branched and especially saturated C₁-C₁₀ alkyl radical optionally substituted with a group OR₁₄,

4) a saturated or unsaturated ring C¹⁹ of 5 to 6 atoms optionally containing at least one heteroatom to form a heterocycle Hy₁₉ and/or optionally comprising a carbonyl function;

ε) R₁₄ is selected from:

1) a hydrogen atom,

2) a linear or branched and especially saturated C₁-C₁₀ alkyl radical;

η) Hy₁₇, Hy₁₈ and Hy₁₉ independently represent a heterocycle of 5 to 6 atoms optionally having from 1 to 4 heteroatoms selected from among N, O and S and combinations thereof and/or comprising a carbonyl function.

According to one preferred embodiment of the invention, the 2-oxyacetamide compounds satisfy one of the formulae (II) to (V) below, or a corresponding salt and/or solvate thereof:

Formula (II):

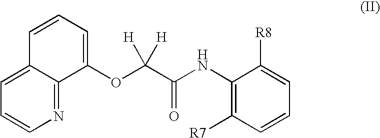

Formula (III):

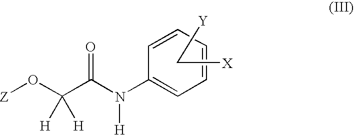

Formula (IV):

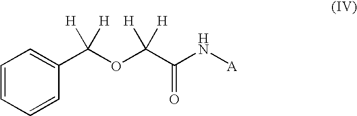

Formula (V): 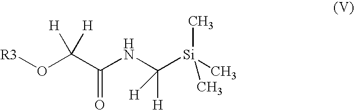
in which:
(i) R7 and R8 independently represent:

1) a hydrogen atom

2) a halogen atom F or Cl,

3) one of the groups CF₃, CN, OR₄, SR₄, NHR₆, NR₆R′₆, OCOR₄, COR₄, CSR₄, COOR₄, SO₂R₄, SiR₄R′₄R″₄ and OCF₃, in which R₄, R′₄ and R″₄ independently represent a hydrogen atom or a C₁-C₂₀ alkyl radical optionally substituted with a phenyl radical and in which R₆ and R′₆ represent a C₁-C₂₀ alkyl radical optionally substituted with a phenyl radical,

4) a C₁-C₂₀ alkyl radical optionally substituted with a group OR₅ in which R5 is a hydrogen or a C₁-C₂₀ alkyl radical,

5) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function;
(ii) Z is one of the following rings and heterocycles: 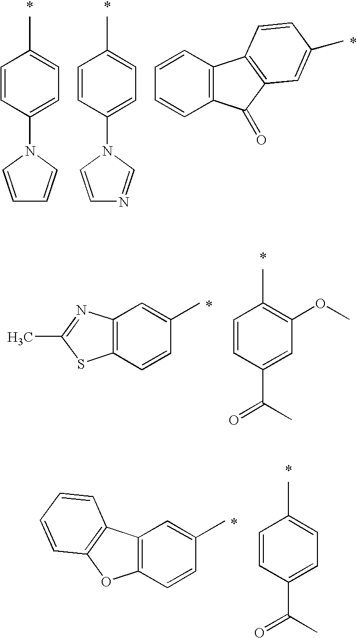
(iii) X and Y independently represent a hydrogen atom or A₂ as defined above for formula (I) or may form a fused ring C¹³ of 4 to 7 atoms and preferably of 5 to 6 atoms, optionally comprising at least one heteroatom selected from N, O and S and combinations thereof, and being optionally substituted with one or more substituents A₂, and optionally fused to another ring C¹⁴; with the proviso that when Z is the dibenzofuran heterocycle, X and Y independently represent:

1) a hydrogen atom,

2) a halogen atom (F or Cl),

3) one of the groups CF₃, CN, OR₉, SR₄, NR₄R′₄, OCOR₄, COR₄, CSR₄, COOR₄, SO₂R₄, SiR₄R′₄R″₄, NO₂ and OCF₃, in which R₄, R′₄ and R″₄ independently represent a hydrogen atom or a C₁-C₂₀ alkyl radical optionally substituted with a phenyl radical and in which R₉ is a hydrogen atom or a C₂-C₂₀ alkyl radical optionally substituted with a phenyl radical,

4) a C₂-C₂₀ alkyl radical optionally substituted with a group OR₅,

5) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function; or when Z is the ring: 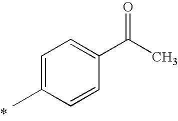
X and Y independently represent:

1) a hydrogen atom, in this case X is different than Y,

2) fluorine,

3) one of the groups OR₁₀, SR₄, NR₄R′₄, OCOR₄, COR₄, CSR₄, COOR₆, SiR₄R′₄R″₄ and OCF₃, in which R₄, R′₄ and R″₄ independently represent a hydrogen or a C₁-C₂₀ alkyl radical optionally substituted with a phenyl radical, in which R₆ is a C₁-C₂₀ alkyl radical optionally substituted with a phenyl radical and in which R₁₀ is a hydrogen atom or a benzyl radical or a C₃-C₂₀ alkyl radical optionally substituted with a phenyl radical,

4) a C₂-C₂₀ alkyl radical optionally substituted with a group OR₅,

5) X and Y may also form a saturated or unsaturated, 5-membered (or 5-atom) ring C¹³ optionally containing one or two oxygen atoms and being optionally fused to another saturated or unsaturated ring C¹⁴;
(iv) A is one of the following three groups: 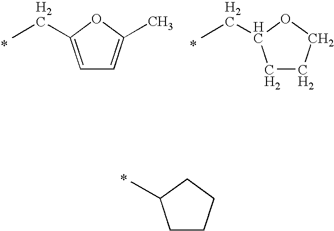
(v) R₃ has the same definition as given for formula (I).

Moreover, the rings C¹¹ and C¹² have the same definitions as above.

To the knowledge of the present inventors, no prior art document describes or suggests that the 2-oxyacetamide compounds of formula (I) or the salts and/or solvated forms thereof have the property of inducing and/or stimulating the growth of human keratin fibers and especially human hair and eyelashes, and/or of retarding their loss, or that these compounds can be administered topically to increase the density of keratin fibers (especially hair and eyelashes).

The effective amount of a compound of formula (I) (salified or non-salified, and solvated or non-solvated) corresponds to the amount of compound required to obtain the desired result (i.e., to increase the density of keratin fibers and especially the hair and the eyelashes, or to promote their growth). One skilled in the art is thus capable of evaluating this effective amount, which depends on the nature of the compound used, the person on whom it is applied and the time of this application.

In the text hereinbelow, and unless otherwise indicated, the amounts of the various ingredients in the composition are given as weight percentages relative to the total weight of the composition.

To provide an order of magnitude, according to the invention, the compound of formula (I) (salified or non-salified, and solvated or non-solvated) or a mixture of compounds of formula (I) and/or of salts thereof may be used in an amount representing from 10⁻³% to 10% of the total weight of the composition, preferably in an amount representing from 10⁻³% to 5% to better still from 10⁻²% to 2% of the total weight of the composition, for example from 0.5% to 2%.

The compositions of the invention may be for cosmetic or pharmaceutical application. The compositions of the invention are preferably for cosmetic use. In addition, the composition must contain a non-toxic, physiologically acceptable medium that can be applied to human skin, including the scalp and the eyelids and to keratin fibers. For the purposes of the invention, the term “cosmetic” means a composition of pleasant appearance, odor and feel.

The compounds of formula (I), salified or non-salified, and solvated or non-solvated, may be formulated into compositions that should be ingested, injected or topically applied to the skin or to keratin fibers (to any area of skin or fibers to be treated).

According to the invention, the compound of formula (I) or a mixture of compounds of formula (I) may be administered orally in an amount of from 0.1 to 300 mg per day, for example from 5 to 10 mg/day.

A preferred composition of the invention is a composition for cosmetic application and in particular for topical application to the skin and keratin fibers, and more especially to the scalp, the hair and the eyelashes.

This composition may be in any known presentation form that is suitable for the mode of use.

For topical application to the skin or keratin fibers, the composition may be in the form of an aqueous, alcoholic or aqueous-alcoholic solution or suspension, or an oily suspension or solution, an emulsion or dispersion of more or less fluid consistency and especially of liquid or semi-liquid consistency, obtained by dispersion of a fatty phase in an aqueous phase (O/W) or conversely (W/O), a solid (O/W) or (W/O) emulsion or dispersion, a more or less fluid or solid aqueous, aqueous-alcoholic or oily gel, a free or compacted powder to be used in unmodified form or to be incorporated into a physiologically acceptable medium, or alternatively microcapsules, microparticles or vesicular dispersions of ionic and/or non-ionic type.

A composition in the form of a foam or alternatively in the form of a spray or aerosol, then comprising a pressurized propellant, is also intended.

It may also be in the form of a lotion, serum, milk, O/W or W/O cream, gel, unguent, ointment, powder, balm, patch, impregnated pad, cake or foam.

In particular, the compositions for application to the scalp or the hair may be in the form of a hair care lotion, for example for daily or twice-weekly application, a shampoo or a hair conditioner, in particular for twice-weekly or weekly application, a liquid or solid scalp cleansing soap for daily application, a hairstyle shaping product (lacquer, hair setting product or styling gel), a treatment mask, a foaming gel or cream for cleansing the hair. It may also be in the form of a hair dye or mascara to be applied with a brush or a comb.

Moreover, for application to the eyelashes or body hairs, the compositions to which the invention relates may be in the form of a pigmented or unpigmented mascara, to be applied with a brush to the eyelashes or alternatively to beard or moustache hair.

For a composition for administration by injection, the composition may be in the form of an aqueous lotion or an oily suspension. For oral administration, the composition may be in the form of capsules, granules, drinkable syrups or tablets.

According to one particular embodiment, the composition according to the invention is in the form of a hair cream or hair lotion, a shampoo, a conditioner for the hair or a mascara for the hair or for the eyelashes.

The amounts of the various constituents according to the invention are those generally used in the fields under consideration. In addition, these compositions are prepared according to the usual methods.

When the composition is an emulsion, the proportion of the fatty phase may range from 2% to 80% by weight and preferably from 5% to 50% by weight relative to the total weight of the composition. The aqueous phase is adjusted as a function of the content of fatty phase and of compound(s) (I) and also of that of the optional additional ingredients, to obtain 100% by weight. In practice, the aqueous phase is from 5% to 99.9% by weight.

The fatty phase may contain fatty or oily compounds that are liquid at room temperature (25° C.) and atmospheric pressure (760 mmHg), which are generally known as oils. These oils may be mutually compatible or incompatible and may form a macroscopically homogeneous liquid fatty phase or a two-phase or three-phase system.

In addition to the oils, the fatty phase may contain waxes, gums, lipophilic polymers or “pasty” or viscous products containing solid parts and liquid parts.

The aqueous phase contains water and optionally an ingredient that is miscible in all proportions with water, for instance C₁ to C₈ lower alcohols such as ethanol or isopropanol, polyols, for instance propylene glycol, glycerol or sorbitol, or alternatively acetone or ether.

The emulsifiers and coemulsifiers used to obtain a composition in emulsion form are those generally included in cosmetics and pharmaceuticals. Their nature also depends on the sense of the emulsion. In practice, the emulsifier and, where appropriate, the coemulsifier are present in the composition in a proportion ranging from 0.1% to 30% by weight, preferably from 0.5% to 20% by weight and better still from 1% to 8% by weight. The emulsion may also contain lipid vesicles and especially liposomes.

When the composition is in the form of an oily solution or gel, the fatty phase may constitute more than 90% of the total weight of the composition.

Advantageously, for a hair application, the composition of the invention is an aqueous, alcoholic or aqueous-alcoholic solution or suspension and preferably a water/ethanol solution or suspension. The alcoholic fraction may represent from 5% to 99.9% to better still from 8% to 80%.

For a topical mascara application, the composition of the invention is especially in the form of a wax-in-water or wax-in-oil dispersion, a gelled oil or an aqueous gel, which may be pigmented or unpigmented.

The compositions of the invention may also comprise other additional ingredients usually employed in the fields under consideration, selected from among aqueous-phase or oily-phase solvents, thickeners or gelling agents, dyes that are soluble in the medium of the composition, solid particles such as fillers or pigments, antioxidants, preservatives, fragrances, electrolytes, neutralizers, film-forming polymers, UV blockers, for instance sunscreens, cosmetic and pharmaceutical active agents with a beneficial effect on the skin or keratin fibers, other than the compounds of formula (I), and mixtures thereof. These additives may be present in the composition in the amounts generally used in cosmetics and dermatology, and especially in a proportion of from 0.01% to 50% to preferably from 0.1% to 20%, for example from 0.1% to 10%, relative to the total weight of the composition. Depending on their nature, these additives may be introduced into the fatty phase, into the aqueous phase and/or into the lipid vesicles and especially liposomes.

Needless to say, one skilled in the art will take care to select the optional additional ingredients and/or the amount thereof such that the advantageous properties of the composition according to the invention, i.e., the inhibition of type-1 15-PGDH and in particular the increase in the density of keratin fibers, are not, or are not substantially, adversely affected by the envisioned addition.

As solvents according to the invention, exemplary are C₂ to C₈ lower alcohols, for instance ethanol, isopropanol, propylene glycol and certain light cosmetic oils, for instance C₆ to C₁₆ alkanes.

As oils according to the invention, exemplary are oils of mineral origin (liquid petroleum jelly or hydrogenated isoparaffin), oils of plant origin (liquid fraction of shea butter, sunflower oil, apricot oil, fatty alcohol or fatty acid), oils of animal origin (perhydrosqualene), synthetic oils (fatty acid ester, purcellin oil), silicone oils (linear or cyclic polydimethylsiloxane, phenyl trimethicone) and fluoro oils (perfluoropolyethers). Waxes that are exemplary include silicone waxes, beeswax, rice wax, candelilla wax, carnauba wax, paraffin wax and polyethylene wax.

As emulsifiers according to the invention, examples include glyceryl stearate, glyceryl laurate, sorbitol stearate, sorbitol oleate, alkyl dimethicone copolyols (with alkyl≧8) and mixtures thereof for a W/O emulsion. Polyethylene glycol monostearate or monolaurate, polyoxyethylenated sorbitol stearate or oleate, and dimethicone copolyols, and mixtures thereof, may also be used for an O/W emulsion.

As hydrophilic gelling agents according to the invention, exemplary are carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents, exemplary are modified clays, for instance Bentones, metal salts of fatty acids, for instance aluminum stearates, hydrophobic-treated silica and ethylcellulose, and mixtures thereof.

As cosmetic or pharmaceutical active agents other than the compound of formula (I), which may be administered according to the invention, exemplary are hydrophilic active agents selected from among proteins or protein hydrolysates, amino acids, polyols, urea, allantoin, sugars and sugar derivatives, water-soluble vitamins, plant extracts (those from Iridacea plants or from soybean) and hydroxy acids such as fruit acids or salicylic acid; and lipophilic active agents such as retinol (vitamin A) and its derivatives, especially an ester (retinyl palmitate), tocopherol (vitamin E) and its derivatives, especially esters (tocopheryl acetate), essential fatty acids, ceramides, essential oils, salicylic acid derivatives, for instance 5-octanoylsalicylic acid, hydroxy acid esters, and phospholipids, for instance lecithin, and mixtures thereof.

According to one particular embodiment of the invention, the compound of formula (I) or a salt and/or solvate thereof may be combined with additional compounds that promote the regrowth and/or limit the loss of keratin fibers (hair or eyelashes). These additional compounds are selected especially from among the lipoxygenase inhibitors as described in EP-648,488, the bradykinin inhibitors described especially in EP-845,700, prostaglandins and derivatives thereof, especially those described in WO 98/33497, WO 95/11003, JP 97-100 091 and JP 96-134 242, prostaglandin receptor agonists or antagonists, the non-prostanoic prostaglandin analogs as described in EP-1,175,891, EP-1,175,890, WO 01/74307, WO 01/74313, WO 01/74314, WO 01/74315 or WO 01/72268, and mixtures thereof.

As other additional active compounds that promote the growth of keratin fibers and/or limit their loss (particularly the hair and the eyelashes), which may be present in the compositions according to the invention, exemplary are vasodilators, anti-androgens, cyclosporins and analogs thereof, anti-microbial and anti-fungal agents, anti-inflammatory agents, and retinoids, alone or in a mixture.

The vasodilators that may be administered are especially potassium-channel agonists, including minoxidil, and also the compounds described in U.S. Pat. Nos. 3,382,247, 5,756,092, 5,772,990, 5,760,043, 5,466,694, 5,438,058 and 4,973,474, cromakalim, nicorandil and diaxozide, alone or in combination.

The anti-androgens that may be administered especially include steroidal or non-steroidal 5α-reductase inhibitors, for instance finasteride and the compounds described in U.S. Pat. No. 5,516,779, cyprosterone acetate, azelaic acid and the salts and derivatives thereof, and the compounds described in U.S. Pat. No. 5,480,913, flutamide, oxendolone, spironolactone, diethylstilbestrol and the compounds described in U.S. Pat. Nos. 5,411,981, 5,565,467 and 4,910,226.

The anti-microbial or anti-fungal compounds may be selected from among selenium derivatives, octopirox, triclocarban, triclosan, zinc pyrithione, itraconazole, asiatic acid, hinokitiol, mipirocine, tetracyclines, especially erythromycin and the compounds described in EP-0,680,745, clinycin hydrochloride, benzoyl peroxide or benzyl peroxide, minocycline and compounds belonging to the imidazole class, such as econazole, ketoconazole or miconazole or salts thereof, nicotinic acid esters, especially including tocopheryl nicotinate, benzyl nicotinate and C₁-C₆ alkyl nicotinates, for instance methyl nicotinate or hexyl nicotinate.

The anti-inflammatory agents may be selected from among steroidal anti-inflammatory agents, for instance glucocorticoids, corticosteroids (for example: hydrocortisone) and non-steroidal anti-inflammatory agents, for instance glycyrrhetinic acid and α-bisabolol, benzydamine, salicylic acid and the compounds described in EP-0,770,399, WO 94/06434 and FR-2,268,523.

The retinoids may be selected from among isotretinoin, acitretin, tazarotene, retinal and adapalene.

As other additional active compounds for promoting the growth and/or limiting the loss of keratin fibers such as the hair and the eyelashes, that may be used in combination with the compounds of formula (I), which may or may not be salified and may or may not be solvated, exemplary are aminexil, 6-0-[(9Z,12Z)octadeca-9,12-dienoyl]hexapyranose, benzalkonium chloride, benzethonium chloride, phenol, estradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharidic or acylhexosaccharidic acids, substituted arylethylenes, N-acylamino acids, flavonoids, ascomycin derivatives and analogs, histamine antagonists, saponins, proteoglycanase inhibitors, estrogen agonists and antagonists, pseudoterines, cytokines, growth factor promoters, IL-1 or IL-6 inhibitors, IL-10 promoters, TNF inhibitors, benzophenones, hydantoin, retinoic acid; vitamins, for instance vitamin D, vitamin B12 analogs and pantothenol; triterpenes, for instance ursolic acid and the compounds described in U.S. Pat. Nos. 5,529,769, 5,468,888 and 5,631,282; anti-pruriginous agents, for instance thenaldine, trimeprazine or cyproheptadine; anti-parasitic agents, in particular metronidazole, crotamiton or pyrethroids; calcium antagonists, for instance cinnarizine, diltiazem, nimodipine, verapamil, alverine and nifedipine; hormones such as oestriol or its analogs, thyroxine and its salts, and progesterone; FP receptor (type-F prostaglandin receptor) agonists such as latanoprost, (5E)-7{(1R,2R,3R,5S)-3,5-dihydroxy-2-[(3R)-3-hydroxy-5-phenylpentyl]cyclopentyl}hept-5-enoic acid, bimatoprost, travoprost, unoprostone and butaprost; mixtures thereof.

Advantageously, the compositions according to the invention comprise at least one 15-PGDH inhibitor as defined above and at least one prostaglandin or prostaglandin derivative, for instance the prostaglandins of series 2 especially including PGF2-α and PGE2 in salt or ester form (for example the isopropyl esters), derivatives thereof, for instance 16,16-dimethyl PGE2,17-phenyl PGE2,16,16-dimethyl PGF2-α, 17-phenyl PGF2-α, prostaglandins of series 1, for instance 11-deoxyprostaglandin E1,1-deoxyprostaglandin E1 in salt or ester form, analogs thereof, especially latanoprost, (5E)-7-{(1R,2R,3R,5S)-3,5-dihydroxy-2-[(3R)-3-hydroxy-5-phenylpentyl]cyclopentyl}hept-5-enoic acid, viprostol, bimatoprost, cloprostenol, travoprost, fluprostenol, cloprostenol, butaprost, unoprostone, misoprostol, and the salts or esters thereof.

The subject compositions preferably contain at least one non-prostanoic EP2 and/or EP4 receptor agonist as described especially in EP-1,175,892.

The compositions comprising at least the compound of formula (I), salified or non-salified, solvated or non-solvated, may be in liposomal form, as described especially in WO 94/22468. Thus, the compound encapsulated in the liposomes may be delivered selectively to the hair follicles.

The compositions according to the invention may be topically applied to the alopecic areas of the scalp and the hair of an individual, and optionally left in contact for several hours and optionally rinsed off.

The compositions containing an effective amount of a compound of formula (I), salified or non-salified, solvated or non-solvated, may, for example, be applied in the evening, kept in contact throughout the night and optionally shampooed out in the morning. These applications may be repeated daily for one or more months according to the needs of the individual.

Advantageously, in the process according to the invention, from 5 μL to 500 μL of a solution or composition as defined above, comprising from 0.001% to 5% of 15-PGDH inhibitor, are topically applied to the areas of the scalp to be cared for or treated.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

Examples of 2-oxyacetamide compounds of formula (II) according to the invention that are representative include the following compounds:

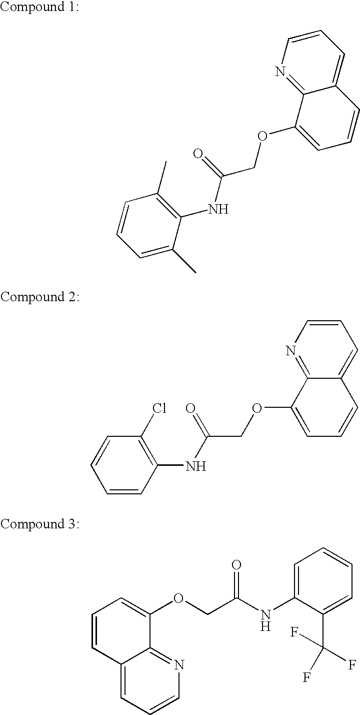

Examples of 2-oxyacetamide compounds of formula (III) according to the invention that are representative include the following compounds:

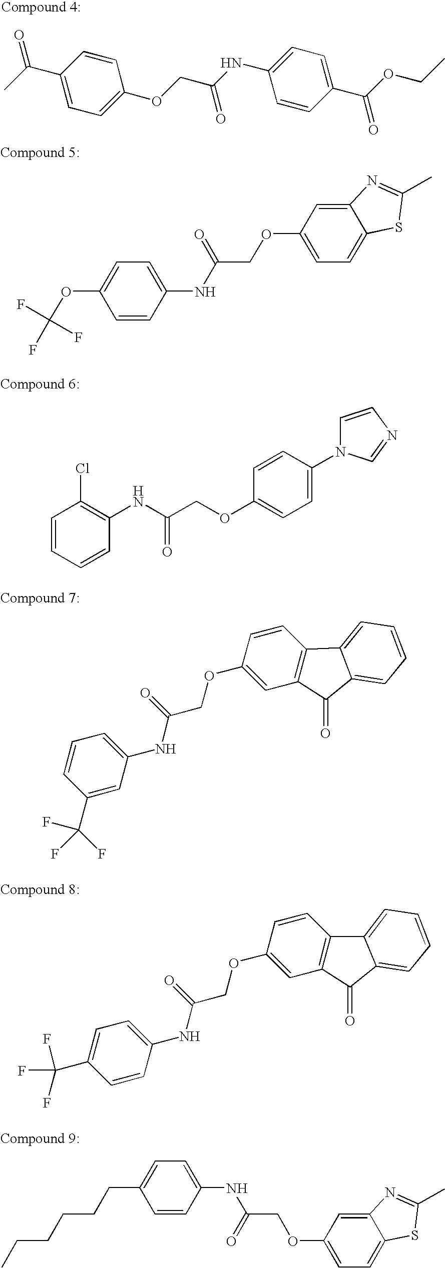

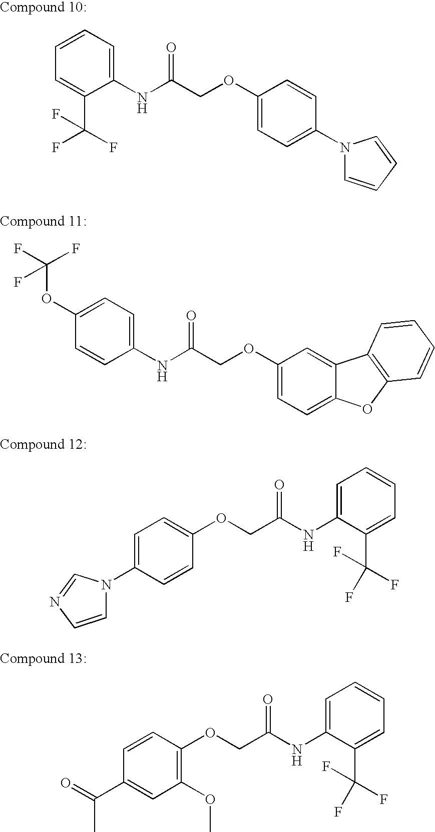

Examples of 2-oxyacetamide compounds of formula (IV) according to the invention that are representative include the following compounds:

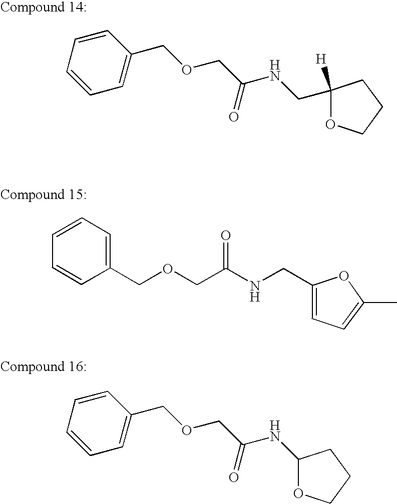

Examples of 2-oxyacetamide compounds of formula (V) according to the invention that are representative include the following compounds:

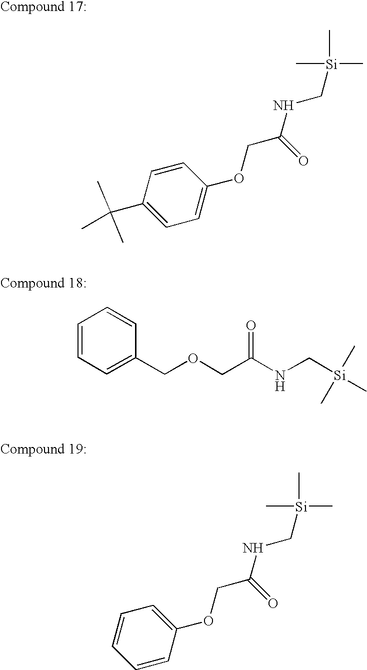

According to another embodiment of the invention, the 2-oxyacetamide compounds satisfy one of the following three formulae:
Compound 20:

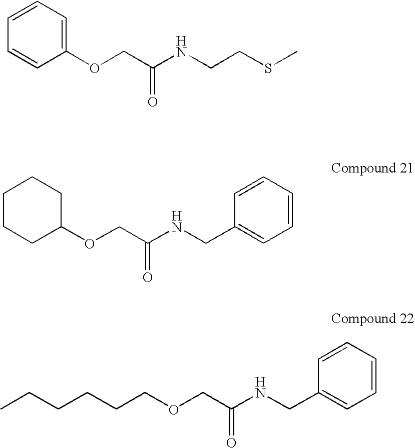

The compounds of formulae (II), (III), (IV) and (V) and Compounds 20, 21 and 22 are novel.

The other compounds to which this invention relates are known per se. They may be prepared in known fashion.

The compounds of formula (I) to which the present invention relates, whether they are novel or known, may be synthesized according to the following reaction scheme:

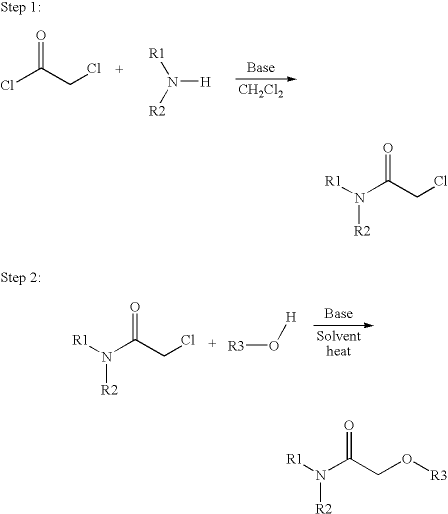

The operating conditions (solvent, base, temperature) for the synthesis of the compounds of formula (I) depend on the starting reagents.

As other examples of 2-oxyacetamide compounds of formula (I) according to the present invention, representative are the following compounds:

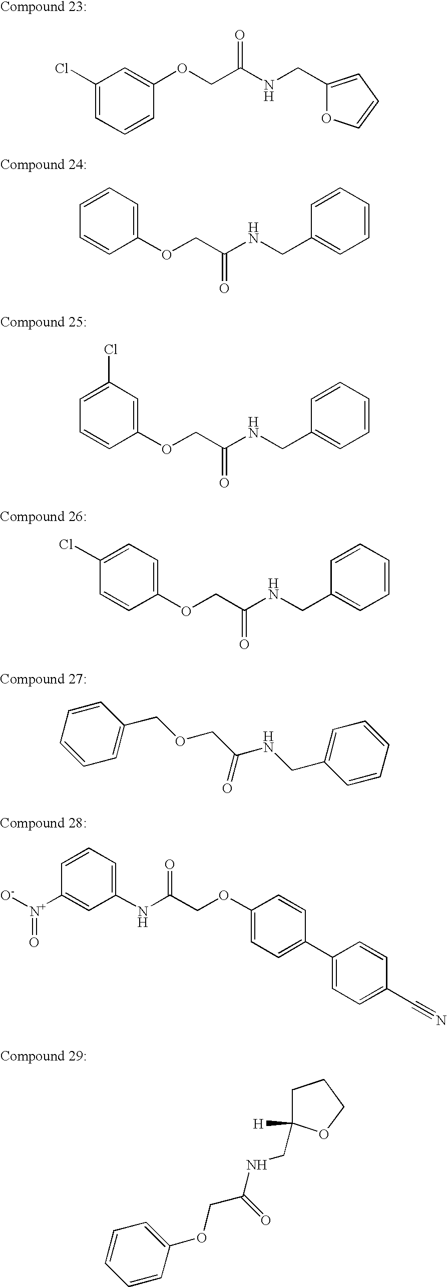

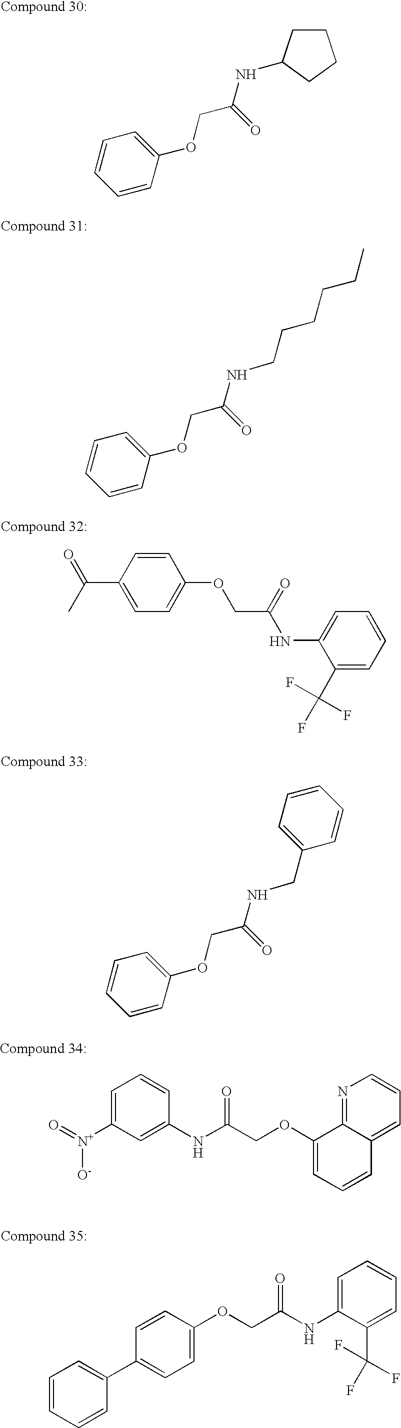

Advantageously, the present invention features the Compounds 2, 3, 5, 6, 8, 9, 10,12,13,18,19, 21, 23, 24, 28, 32, 34 and 35 and in particular novel Compounds 2, 3, 5, 6, 8, 9, 10,12,13,18,19 and 21, and Compounds 23 and 24. Preferably, this invention features the novel Compounds 2, 3,12,13 and 21 and also Compounds 23 and 24.

Non-limiting examples of synthesis of compounds in accordance with the invention will now be given:

### Example 1

### Reaction Scheme for the Synthesis of Compound 21

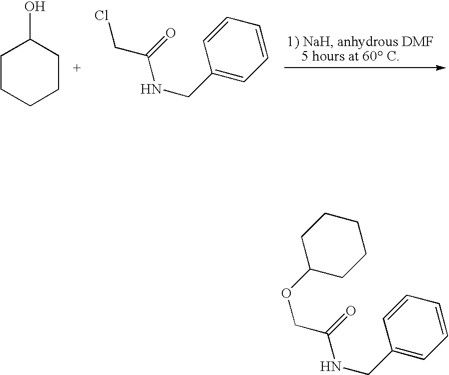

Procedure:

0.63 ml of freshly distilled cyclohexanol (6 mmol) are introduced into a three-necked flask under a flow of argon and diluted in 10 ml of anhydrous dimethylformamide. 60% sodium hydride (0.24 g; 6 mmol) is added portionwise to the reaction medium. The reaction mixture is then stirred at room temperature for 1 hour and a solution of N-benzyl-2-chloroacetamide (1 g; 5.44 mmol) in 10 ml of anhydrous DMF is then added. The medium is maintained at a temperature of 60° C. for 5 hours. The reaction mixture is concentrated to the maximum and then diluted with 100 ml of dichloromethane. The organic phase is washed with water (twice 50 ml) and then with saturated sodium chloride solution. The organic phase is dried over sodium sulfate, filtered and then concentrated to the maximum. The crude product is taken up in a minimum amount of ethyl ether and stirred for 30 minutes. The solid is removed and the filtrate is purified on silica gel (eluent:dichloromethane). After distilling off the solvent and drying under vacuum in the presence of phosphorous pentoxide, a beige-colored solid (0.36 g, in a yield (yld) of 24%) is obtained.

The analyses of the product (Nuclear Magnetic Resonance and Mass Spectrum) are in accordance with formula 21.

### Example 2

### Reaction Scheme for the Synthesis of Compound 22

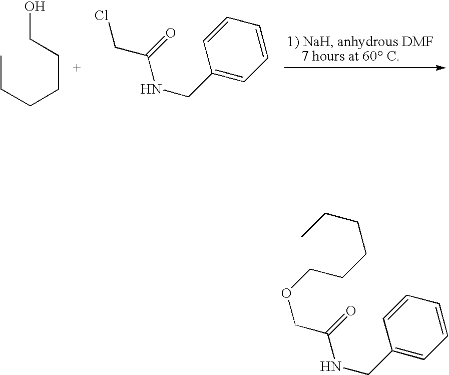

Procedure:

0.7 ml of anhydrous hexanol (5.44 mmol) is diluted in 20 ml of anhydrous DMF in a 50 ml three-necked flask equipped with a condenser and a thermometer, and under a flow of nitrogen. 60% sodium hydride (0.24 g; 6 mmol) is added portionwise to the reaction medium. After addition, the reaction medium is stirred at room temperature for 2 hours and a solution of N-benzyl-2-chloroacetamide (1 g; 5.44 mmol) in 10 ml of anhydrous DMF is then added. The medium is maintained at a temperature of 60° C. for 7 hours. The reaction mixture is concentrated to the maximum and then diluted with 100 ml of dichloromethane. The organic phase is washed with water (twice 50 ml) and then with saturated sodium chloride solution. The organic phase is dried over sodium sulfate, filtered and then concentrated to the maximum. The crude product is taken up in a minimum amount of ethyl ether and stirred for 30 minutes. The solid is removed and the filtrate is purified on silica gel (eluent:dichloromethane). After evaporating off the solvent and drying under vacuum, a yellow oil is obtained (18.5 mg, yld=2%).

The analyses of the product (NMR and Mass Spectrum) are in accordance with those expected for Compound 22.

### Example 3

### Reaction Scheme for the Synthesis of Compound 23

Compound 23 is prepared in two steps.

Step 1: Synthesis of the chloroacetamide (a):

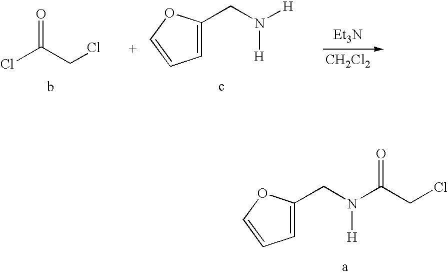

40 ml of furfurylamine (c), 500 ml of dichloromethane and 69.9 ml of triethylamine (abbreviated to Et₃N) are introduced into a three-necked flask. After cooling the reaction medium to 10° C., 37.1 ml of chloroacetyl chloride (b) diluted in 100 ml of dichloromethane are added dropwise while maintaining the temperature below 15° C. The reaction medium is then stirred for 3 hours at room temperature. Water is then added. The organic phase is washed with water (twice 100 ml), then with 1 N dilute hydrochloric acid solution (twice 50 ml), again with water and then with saturated sodium chloride solution. The organic phase is washed over sodium sulfate and then filtered and concentrated to give 66 g of a brown solid (compound a, yield=84%).

Analysis of product (a):

Mass Spectrometry (MS): the quasi-molecular ions (MH)⁺, (MNa)⁺ of the expected molecule C₇H₈CINO₂ are mainly detected.

Nuclear Magnetic Resonance (NMR): ¹H(DMSO, 400 MHz) δ ppm: 8.66 (1H, broad s, NH); 7.58 (1H, dd, Hₐᵣ); 6.40 (1H, dd Hₐᵣ); 6.27 (1H, dd, Hₐᵣ); 4.30 (2H, d, CH₂); 4.09 (2H, s, CH₂).

broad s=broad singlet

d=doublet

dd=doubled doublet

These results are in accordance with the formula of compound (a).

Step 2: Synthesis of Compound 23 ((2-(3-chlorophenoxy)-N-furan-2-ylmethylacetamide:

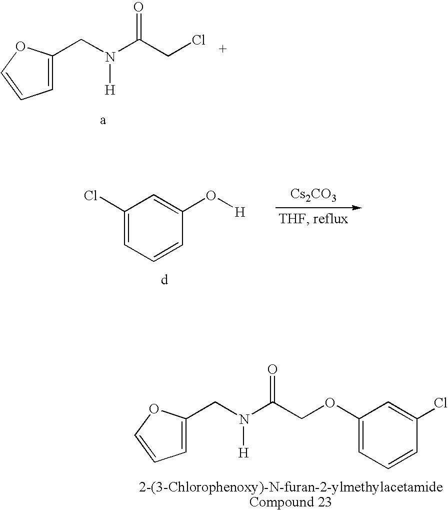

1 g of 3-chlorophenol (d), 20 ml of tetrahydrofuran (THF) and 1.9 g of cesium carbonate are introduced into a three-necked flask. A solution 1.34 g of compound (a) prepared above in 20 ml of dichloromethane (DCM) is then added. The organic medium is refluxed for 5 hours; water is then added. The organic phase is washed with water (twice 100 ml), with 1 N dilute sodium hydroxide solution (twice 50 ml), again with water (twice 100 ml) and then with saturated sodium chloride solution. The organic phase is dried over sodium sulfate and then filtered and concentrated to give 1.5 g of a light-brown solid (Compound 23, yield=73%).

Analysis of the product obtained:

NMR: ¹H(DMSO, 400 MHz) δ ppm: 8.59 (1H, t, NH); 7.56 (1H, bs, H—Ar); 7.32 (1H, m, H—Ar); 7.04 (1H, d, H—Ar); 7.02 (1H, dd, H—Ar); 6.94 (1H, dd, H—Ar); 6.38 (1H, m, H—Ar); 6.21 (1H, m, H—Ar); 4.57 (2H, s, CH₂); 4.33 (2H, d, CH₂).

s=singlet

broad s=broad singlet

d=doublet

dd=doubled doublet

t=triplet

m=multiplet

H—Ar=aromatic hydrogen

Mass Spectrometry: the quasi-molecular ion (MH)⁺ of the expected molecule C₁₃H₁₂ClNO₃ is mainly detected.

These results are in accordance with the formula of Compound 23.

### Examples 4, 5 and 6

### Reaction Scheme for the Synthesis of Compounds 24, 25 and 26

These compounds were prepared according to the same process as for Compound 21, replacing the cyclohexane with, respectively, phenol for Compound 24, 3-chlorophenol for Compound 25, and 4-chlorophenol for Compound 26.

The mass spectra (+ and −ESU) are in accordance with the expected structures.

### Examples 7, 8 and 9

### Reaction Scheme for the Synthesis of Compounds 1, 2 and 3 of General Formula (II)

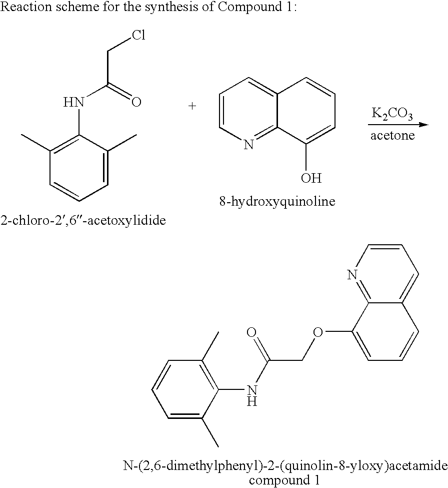

Procedure:

A suspension of 28 mg of potassium carbonate in 1 ml of acetone is introduced into a reaction tube equipped with a magnetic bar. A solution of 6.4 mg of 8-hydroxyquinoline (CAS: 148-24-3; Fluka reference: F55080) in 0.5 ml of acetone and a solution of 7.9 mg of 2-chloro-2′,6′-acetoxylidide (CAS: 1131-01-7) in 0.5 ml of acetone are then added. The medium is diluted with 1 ml of acetone and the tube is stoppered and refluxed for 24 hours. The reaction medium is cooled to room temperature and filtered through a sinter funnel, and the solid is rinsed with 2.5 ml of acetone. The filtrate is concentrated under vacuum, taken up in 300 μl of a 9/1 dichloromethane/methanol mixture and added to a suspension of 80 mg of PL-TBD resin (1,3,4,6,7,8-hexahydro-2H-pyrimido[1,2-a]pyrimidine resin charged at 2.7 mmol/g) in 1.5 ml of dichloromethane. The medium is left for 24 hours and then filtered through a sinter funnel and concentrated. Compound 1 (N-(2,6-dimethylphenyl)-2-(quinolin-8-yloxy)acetamide) is obtained in the form of a brown solid (9.2 mg) in a yield of 75%.

Analysis:

Mass Spectrometry: (+ and −ESI): (M+H)⁺=307 and (M+Na)⁺=330

Compounds 2 and 3 were obtained in a similar manner by reacting 8-hydroxyquinoline (CAS: 148-24-3; Fluka reference: F55080) with the following commercial chloroacetamides according to the same procedure:

N-(2-chlorophenyl)-2-chloroacetamide (3289-76-7) for Compound 2 (N-(2-chlorophenyl)-2-quinolin-8-yloxy)acetamide) obtained in the form of a brown solid (7.4 mg) in a yield of 59%.

N-(2-chloroacetyl)-2-(trifluoromethyl)aniline (3792-04-9) for Compound 3 (2-(quinolin-8-yloxy)-N-(2-trifluoromethylphenyl)acetamide) obtained in the form of an orange solid (6.5 mg) in a yield of 47%.

### Examples 10 to 19

### Reaction Scheme for the Synthesis of Compounds 4 to 13 of General Formula (III)

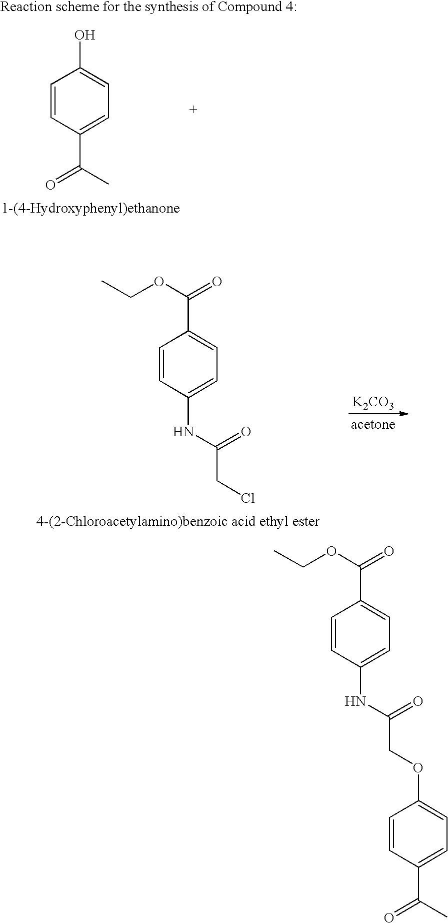 4-[2-(4-Acetylphenoxy)acetylamino]benzoic acid ethyl
Compound 4

The procedure is the same as that described for the synthesis of the compounds of general formula (II).

Thus, Compound 4 is obtained in the form of white crystals (11.2 mg) in a yield of 82% by reaction from 1-(4-hydroxyphenyl)ethanone (4-hydroxyacetophenone CAS: 99-93-4) and ethyl 4-(2-chloroacetamido)benzoate (4-(2-chloroacetylamino)benzoic acid ethyl ester CAS: 26226-72-2).

Similarly, Compounds 5, 6, 7, 8, 9, 10, 11, 12 and 13 are obtained according to the Table below:

| | Starting | Starting | Com- | | Appear- | Mass |
|---|---|---|---|---|---|---|
| Structure | phenol | chloride | pound | Name | ance | (yield) |
| 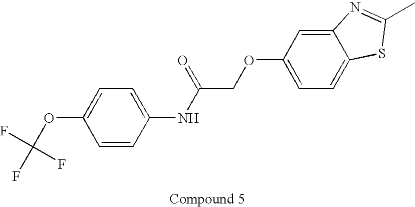 | 2-methyl- 5-benzo- thiazolol | N-(chloro- acetyl)-4- (trifluoro- methoxy)- aniline | 5 | 2-(2-Methyl- benzothiazol-5- yloxy)-N-(4-tri- fluoromethoxy- phenyl) acetamide | white solid | 5.3 mg (35%) |
| 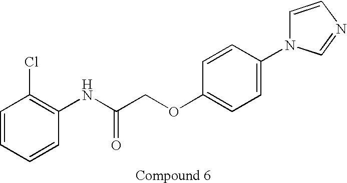 | 4-(Imidaz- ol-1-yl) phenol | N-(2- chloro- phenyl)-2- chloro- acetamide | 6 | N-(2-Chloro- phenyl)-2-(4- imidazol-1-yl- phenoxy)- acetamide | beige solid | 10.3 mg (79%) |
| 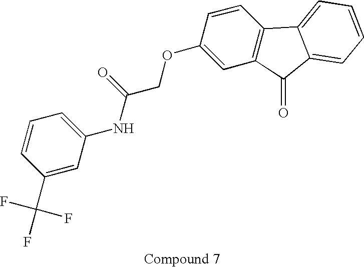 | 2- Hydroxy- 9-Fluo- renone | N- (choloro- acetyl)-3- (trifluoro- methyl)- aniline | 7 | 2-(9-Oxo-9H- fluoren-2- yloxy)-N-(3-tri- fluoromethyl- phenyl) acetamide | orange solid | 6.6 mg (41%) |
| 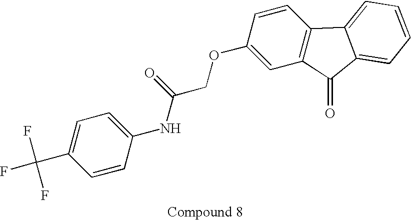 | 2- Hydroxy- 9-Fluo- renone | N- chloro- acetyl-4- (trifluoro- methyl) aniline | 8 | 2-(9-Oxo-9H- fluoren-2- yloxy)-N-(4-tri- fluoromethyl- phenyl) acetamide | yellow solid | 2.4 mg (15%) |
| 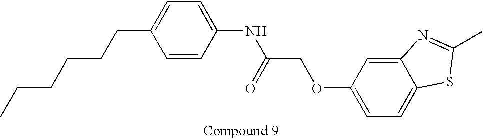 | 2- methyl-5- benzo- thiazolol | | 9 | N-(4-Hexyl- phenyl)-2-(2- methyl-benzo- thiazol-5- yloxy) acetamide | beige solid | 5.1 mg (33%) |
| 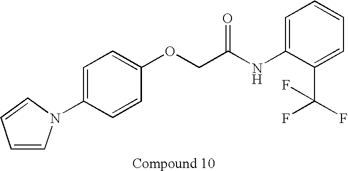 | 4-(1H- Pyrrol- 1-yl) phenol | N-(chloro- acetyl)-2- (trifluoro- methyl) aniline | 10 | (2-(4-Pyrrol-1- yl-phenoxy)-N- (2-trifluoro- methyl-phenyl) acetamide | orange crystals | 4.7 mg (33%) |
| 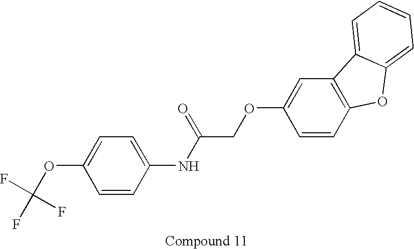 | 2- hydroxy dibenzo- furan | N-(chloro- acetyl)-4- (trifluoro- methoxy)- aniline | 11 | 2-(Dibenzo- furan-2-yloxy)- N-(4-trifluoro- methoxy- phenyl) acetamide | white solid | 4.8 mg (30%) |
| 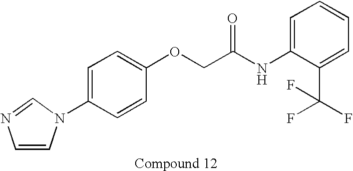 | 4-(Imidaz- ol-1-yl) phenol | N-(chloro- acetyl)-2- (trifluoro- methyl) aniline | 12 | 2-(4-imidazol- 1-yl-phenoxy)- N-(2-trifluoro- methyl-phenyl) acetamide | ochre solid | 4.9 mg (34%) |
| 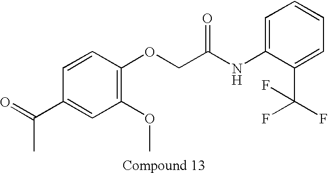 | 4-hyroxy- 3- methoxy- aceto- phenone, 97% | N-(chloro- acetyl)-2- (trifluoro- methyl) aniline | 13 | 2-(4-Acetyl-2- methoxy- phenoxy)-N-(2- trifluoro- methyl-phenyl) acetamide | yellow solid | 7 mg (48%) |

### Examples 20, 21 and 22

### Reaction Scheme for the Synthesis of Compounds 14, 15 and 16 of General Formula (IV)

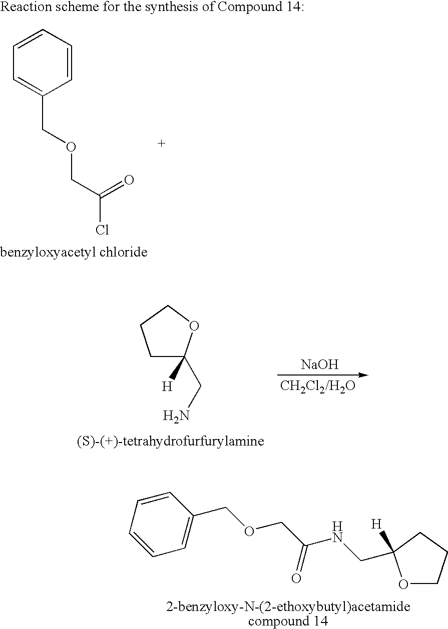

Procedure:

A solution of 9.6 mg of benzyloxyacetyl chloride (CAS: 19810-31-2; Aldrich reference: 30,101-9) in 0.5 ml of dichloromethane and a solution of 5.2 mg of (S)-(+)-tetrahydrofurfurylamine (CAS: 7175-81-7) in 0.5 ml of 1N sodium hydroxide are introduced into a reaction tube equipped with a magnetic bar. The medium is diluted with 0.5 ml of dichloromethane, and 1 ml of 1 N sodium hydroxide solution is then introduced therein. The reaction medium is maintained under magnetic stirring for 24 hours and then thrown onto a liquid-liquid extraction cartridge (Chem Elut Varian) and the organic phase is evaporated. Compound 14 (2-benzyloxy-N-(tetrahydrofuran-2-ylmethyl)acetamide) is obtained in a yield of 72% (7.2 mg).

Analysis:

Mass Spectrometry: ESI (+ and −): (M+H)⁺=250 and (M+Na)⁺=272

Compounds 15 and 16 are obtained according to same procedure. The data are collated in the following Table:

| | Starting | Starting | Com- | | | Mass |
|---|---|---|---|---|---|---|
| Structure | acid chloride | amine | pound | Name | Appearance | (yield) |
| Compound 15 | | | | | | |
| 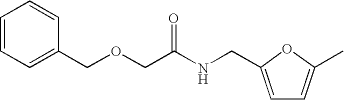 | benzyloxyacetyl chloride | 5-Methyl- furfuryl- amine | 15 | 2-Benzyloxy-N-(5-methyl- furan-2-ylmethyl)- acetamide | white solid | 7.9 mg (76%) |
| Compound 16 | | | | | | |
| 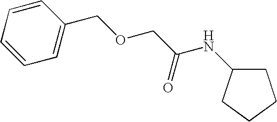 | benzyloxyacetyl chloride | cyclo- pentyl- amine | 16 | 2-Benzyloxy-N- cyclopentylacetamide | white solid | 8 mg (86%) |

### Examples 23 to 25

### Reaction Scheme for the Synthesis of Compounds 17, 18 and 19 of General Formula (V)

The procedure is the same as that described for the synthesis of the compounds of general formula (IV).

Compounds 17, 18 and 19 are obtained according to the data collated in the following Table:

| | Starting | | Com- | | | Mass |
|---|---|---|---|---|---|---|
| Structure | acid chloride | Starting amine | pound | Name | Appearance | (yield) |
| Compound 17 | | | | | | |
| 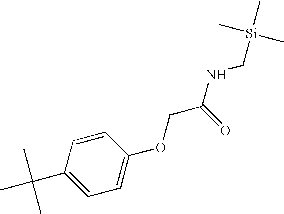 | 4-tert-butyl- phenoxyacetyl chloride | (trimethylsilyl) methylamine | 17 | 2-(4-tert-Butyl-phenoxy)- N-trimethylsilanylmethyl- acetamide | white solid | 50 mg (85%) |
| Compound 18 | | | | | | |
| 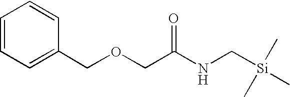 | benzyloxyacetyl chloride | (trimethylsilyl) methylamine | 18 | 2-Benzyloxy-N- trimethylsilanylmethyl- acetamide | white solid | 7.5 mg (75%) |
| Compound 19 | | | | | | |
| 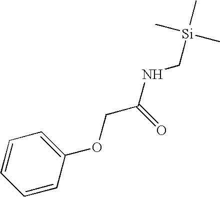 | Phenoxyacetyl chloride | (trimethylsilyl) methylamine | 19 | 2-Phenoxy-N- trimethylsilanylmethyl- acetamide | white solid | 7.1 mg (75%) |

### Example 26

### Reaction Scheme for the Synthesis of Compound 20

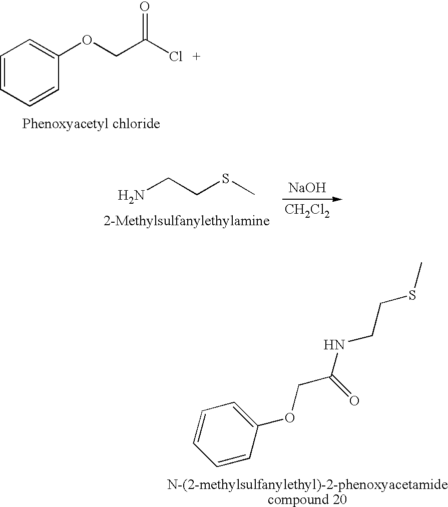

The procedure is the same as that used for the synthesis of the compounds of general formula (IV). Compound 20 is obtained in the form of a white solid (7.8 mg) in a yield of 87%.

### Example 27

### Demonstration of the Specific Inhibitory Properties on 15-PGDH of the Compounds of Formula (I)

1) Test on type-1 15-PGDH:

The enzyme 15-PGDH is obtained as described in the FR 02/05067 filed by the assignee hereof, as a suspension in a medium adapted to a concentration of 0.3 mg/ml and then frozen at −80° C. For the purposes of the test, this suspension is thawed and stored in ice.

Separately, a pH 7.4 100 mM Tris buffer is prepared, containing 0.1 mM of dithiothreitol (D5545, Sigma-Aldrich, L'isle D'Abeau Chesne, BP 701, 38297, Saint Quentin Fallavier), 1.5 mM of β-NAD (N6522, Sigma-Aldrich, L'isle D'Abeau Chesne, BP 701, 38297, Saint Quentin Fallavier), 50 μM of prostaglandin E₂ (P4172, Sigma-Aldrich, L'isle D'Abeau Chesne, BP 701, 38297, Saint Quentin Fallavier).

0.965 ml of this buffer (brought to 37° C. beforehand) is introduced into the cuvette of a spectrophotometer (Perkin-Elmer, Lambda 2) thermostatically maintained at 37° C., the measuring wavelength of which is set at 340 nm. 0.035 ml of enzymatic suspension at 37° C. is introduced into the cuvette concomitantly with the recording (corresponding to an increase in the optical density at 340 nm). The maximum reaction rate is recorded.

The test values (containing the compounds of formula (I)) are compared with the control value (without compound (I)); the results indicated represent either the percentage of inhibition of the enzymatic activity of 15-PGDH for a given concentration of compound of formula (I), or the concentration at which the compound of formula (I) reduces by 50% the enzymatic activity of 15-PGDH, i.e., IC_{50dh}.

2) Test on PGF synthase:

The enzyme PGFS is obtained as described in FR-A-02/05067, at a concentration of 0.5 mg/mL, as a suspension in a suitable medium, and blocked at −80° C. For the purposes of the test, this suspension is thawed and stored in ice.

In parallel, a 100 mM, pH 6.5 Tris buffer containing 20 μM of 9,10-phenanthrenequinone* (P2896, Sigma-Aldrich, L'isle D'Abeau Chesne, BP 701, 38297, Saint Quentin Fallavier) and 100 μM of β-NADPH(N1630, Sigma-Aldrich, L'isle D'Abeau Chesne, BP 701, 38297, Saint Quentin Fallavier) is prepared in a brown flask (protected from light).
*A stock solution with a titer of 1 mM is prepared in absolute ethanol and brought to 40° C.; the flask is placed in an ultrasound cuvette to facilitate the dissolution of the product.

0.950 mL of this buffer (brought to 37° C. beforehand) is introduced into the cuvette of a spectrophotometer (Perkin-Elmer, Lambda 2) thermostatically maintained at 37° C., the measuring wavelength of which is set at 340 nm. 0.05 mL of enzymatic suspension at 37° C. is introduced into the cuvette concomitantly with the recording (corresponding to a reduction in the optical density at 340 nm). The maximum reaction rate is recorded.

The test values (containing compound (I)) are compared with the control value (without compound (I)); the results indicated represent either the percentage of inhibition of the enzymatic activity of PGFS for a given concentration of compound of formula (I), or the concentration at which the compound of formula (I) reduces the enzymatic activity of PGFS by 50%, namely IC_{50fs}.

The results are reported in the table below:

| | | % inhibition |
|---|---|---|
| | | of 15-PGDH |
| Compound | Structure | at 100 μM |
| 1 | 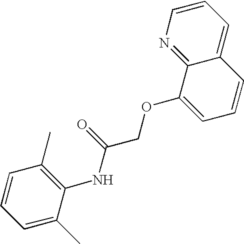 | 49 |
| 2 | 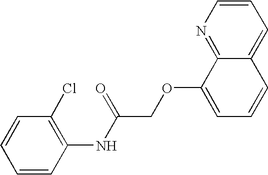 | 115 |
| 3 | 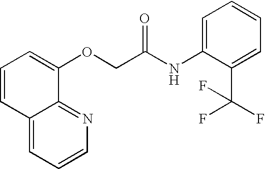 | 128 |
| 4 | 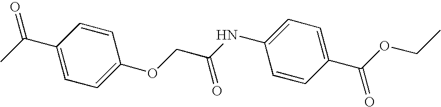 | 49 |
| 5 | 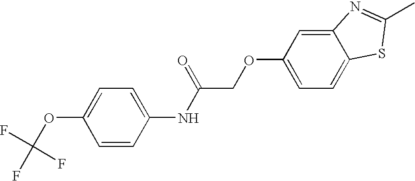 | 53 |
| 6 | 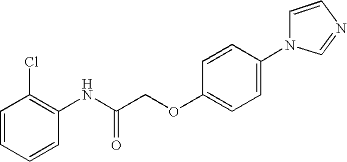 | 54 |
| 7 | 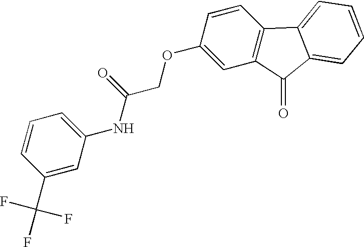 | 61 |
| 8 | 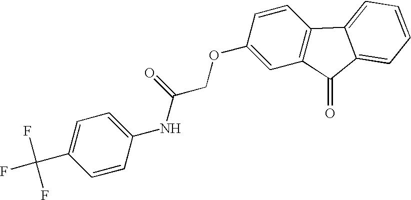 | 65 |
| 9 | 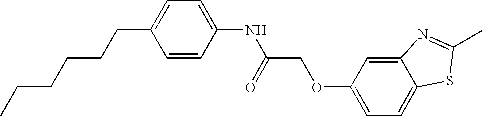 | 69 |
| 10 | 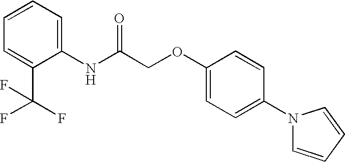 | 71 |
| 11 | 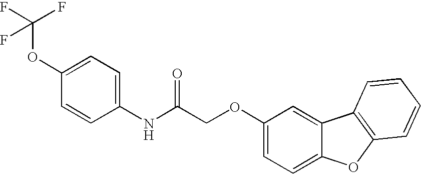 | 74 |
| 12 | 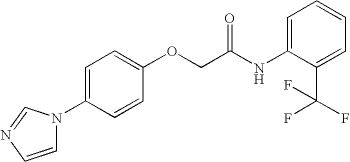 | 125 |
| 13 | 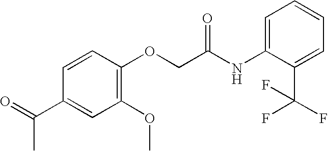 | 126 |
| 14 | 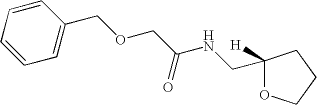 | 50 |
| 15 | 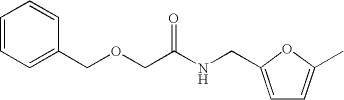 | 51 |
| 16 | 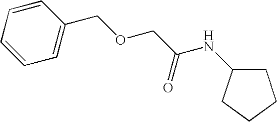 | 76 |
| 17 | 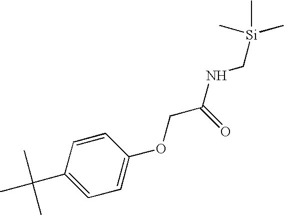 | 50 |
| 18 | 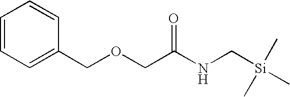 | 88 |
| 19 | 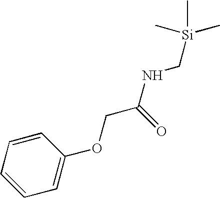 | 95 |
| 20 | 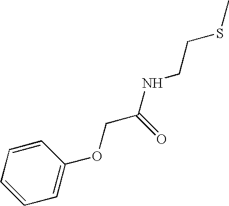 | 85 |
| 27 | 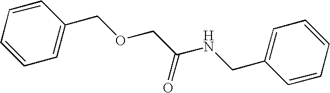 | 48 |
| 28 | 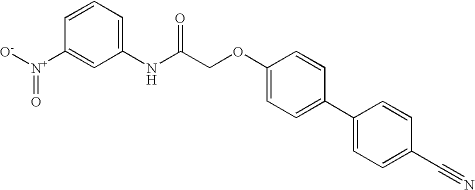 | 54 |
| 29 | 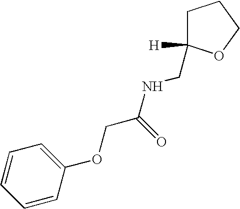 | 54 |
| 30 | 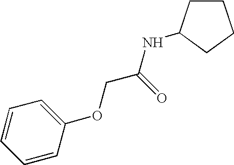 | 56 |
| 31 | 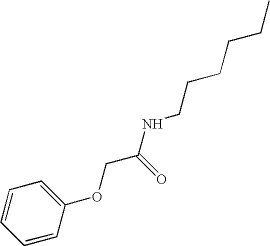 | 57 |
| 32 | 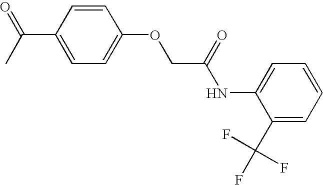 | 59 |
| 33 | 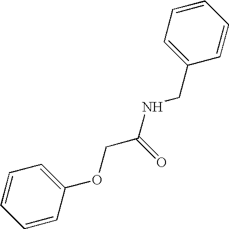 | 61 |
| 34 | 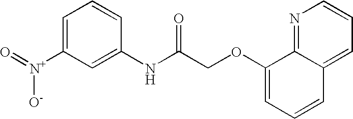 | 69 |
| 35 | 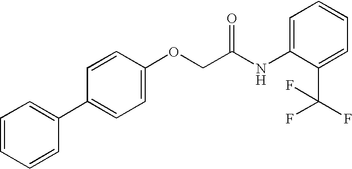 | 70 |

Moreover, Compounds 1 to 20 and 27 to 35 do not inhibit the enzyme PGFS at 100 μM.

| | | Inhibition | | Inhibition | |
|---|---|---|---|---|---|
| | | of 15-PGDH | | of PGFS | |
| | | | % inhibition | | % inhibition |
| Compound | Structure | IC₅₀ | at 50 μM | IC₅₀ | at 50 μM |
| 21 | 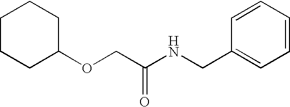 | — | 63 | - | 24 |
| 22 | 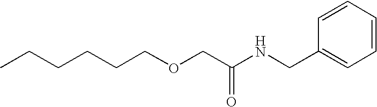 | — | 47 | — | — |
| 23 | 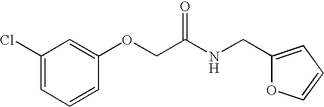 | 16 μM | 80 | >50 μM | 25 |
| 24 | 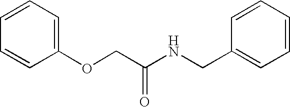 | — | 55 | — | 4 |
| 25 | 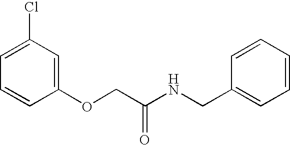 | — | 50 | — | — |
| 26 | 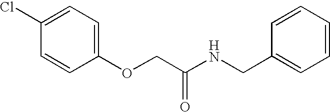 | — | 30 | — | — |

From these tables, it is clearly seen that the compounds of formula (I) are inhibitors of type-1 15-PGDH, and do so selectively compared with the inhibition of PGFS.

The compositions below are formulated via the usual techniques commonly employed in the cosmetic or pharmaceutical field.

### Example 28

Hair lotion:

| | | |
|---|---|---|
| Compound 23 | 1.00 | g |
| Propylene glycol | 30.00 | g |
| Ethyl alcohol | 40.00 | g |
| Water | qs 100.00 | g |

This lotion is applied to the scalp, once or twice a day, at a rate of 1 ml per application, by massaging the scalp gently to make the active agent penetrate. The hair is then air-dried. This lotion makes it possible to retard the loss and to promote the regrowth of the hair. It also allows the appearance of the hair to be improved.

### Example 29

Hair lotion:

| | | |
|---|---|---|
| Compound 21 | 1.00 | g |
| Propylene glycol | 30.00 | g |
| Ethyl alcohol | 40.00 | g |
| Water | qs 100.00 | g |

This lotion is applied to the scalp, once or twice a day, at a rate of 1 ml per application, by massaging the scalp gently to make the active agent penetrate. The hair is then air-dried. This lotion makes it possible to retard the loss and to promote the regrowth of the hair. It also allows the appearance of the hair to be improved.

### Example 30

Wax/water mascara:

| | |
|---|---|
| Beeswax | 6.00% |
| Paraffin wax | 13.00% |
| Hydrogenated jojoba oil | 2.00% |
| Water-soluble film-forming polymer | 3.00% |
| Triethanolamine stearate | 8.00% |
| Compound 23 | 1.00% |
| Black pigment | 5.00% |
| Preservative | qs |
| Water | qs 100.00% |

This mascara is applied to the eyelashes like a standard mascara with a mascara brush.

### Example 31

Hair lotion:

| | | |
|---|---|---|
| Compound 21 | 0.10 | g |
| Latanoprost | 0.10 | g |
| Propylene glycol | 30.00 | g |
| Ethyl alcohol | 40.00 | g |
| Water | qs 100.00 | g |

This lotion is applied to the scalp, once or twice a day, at a rate of 1 ml per application, by massaging the scalp gently to make the active agent penetrate. The hair is then air-dried. This lotion makes it possible to retard the loss and to promote the regrowth of the hair. It also allows the appearance of the hair to be improved.

### Example 32

Hair lotion:

| | | |
|---|---|---|
| (5E)-7-{(1R,2R,3R,5S)-3,5-Dihydroxy- | 0.10 | g |
| 2-[(3R)-3-hydroxy-5-phenylpentyl]cyclopentyl} | | |
| hept-5-enoic acid | | |
| Compound 21 | 0.10 | g |
| Propylene glycol | 30.00 | g |
| Ethyl alcohol | 40.00 | g |
| Water | qs 100.00 | g |

This lotion is applied to the scalp, once or twice a day, at a rate of 1 ml per application, by massaging the scalp gently to make the active agent penetrate. The hair is then air-dried. This lotion makes it possible to retard the loss and to promote the regrowth of the hair. It also allows the appearance of the hair to be improved.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for inducing and/or stimulating the growth of keratin fibers and/or for retarding the loss and/or increasing the density thereof and/or for caring for and/or making up said keratin fibers, comprising administering to a mammalian organism in need of such treatment, for such period of time as required to elicit the desired response, a thus effective amount of at least one 2-oxyacetamide compound of formula (I) or a salt and/or solvate thereof: 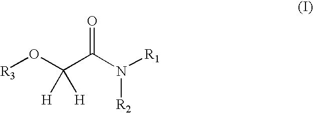
in which:
a) R₁ and R₂ are independently selected from:
1) a hydrogen atom, with R₁ different than R₂,
2) a C₁-C₂₀ alkyl radical optionally substituted with at least one substituent A₁,
3) a hydrocarbon-based ring C¹ of 3 to 7 atoms optionally fused to at least one ring C² of 4 to 7 atoms, the ring C² optionally containing at least one heteroatom to form a heterocycle Hy₂, these rings C¹ and C² optionally comprising a carbonyl or thiocarbonyl function and/or being substituted with at least one substituent A₂,
4) a heterocycle Hy₁, optionally fused to a ring C² of 4 to 7 atoms, the ring C² optionally containing at least one heteroatom to form a heterocycle Hy₂, these rings Hy₁, and C² optionally comprising a carbonyl or thiocarbonyl function and/or being substituted with at least one substituent A₁,
5) one of the groups C(═NR)R′, C(═NR)NR′R″, COR, CSR, COOR, CONRR′, SO₂R or SO₂NRR′;
b) R₃ is selected from:
1) a C₁-C₂₀ alkyl radical optionally substituted with at least one substituent A₁,
2) a hydrocarbon-based ring C³ of 3 to 7 atoms optionally fused to at least one ring C⁴ of 4 to 7 atoms optionally containing at least one heteroatom to form a heterocycle Hy₄, these rings C³ and C⁴ optionally comprising a carbonyl or thiocarbonyl function and/or being substituted with at least one substituent A₂,
3) a heterocycle Hy₃ selected from among pyrrole, furan, thiophene and pyrazole rings and optionally fused to a ring C⁵ constituting a phenyl, a pyridine or a pyrimidine, the heterocycle Hy₃ and the ring C⁵ being optionally substituted with at least one substituent A₁,
4) a heterocycle Hy₅ different than Hy₃ and optionally fused to a ring C⁶ of 4 to 7 atoms, these rings Hy₅ and C⁶ optionally comprising a carbonyl or thiocarbonyl function and/or being substituted with at least one substituent A₁, this ring C⁶ optionally containing at least one heteroatom to form a heterocycle Hy₆;
c) R, R′ and R″ are independently selected from:
1) a hydrogen atom,
2) a C₁-C₂₀ alkyl radical optionally substituted with at least one substituent A₁,
3) a ring C⁷ of 4 to 7 atoms optionally containing at least one heteroatom to form a heterocycle Hy₇ and/or being optionally fused to a ring C⁸ of 4 to 7 atoms optionally containing at least one heteroatom to form a heterocycle Hy₈, the rings C⁷ and C⁸ being optionally substituted with at least one substituent A₁ and optionally comprising a carbonyl or thiocarbonyl function;
d) A₁ is selected from:
1) a halogen atom,
2) a C₁-C₂₀ alkyl radical optionally substituted with a group OR₅,
3) a ring C⁹ of 4 to 15 atoms optionally containing at least one heteroatom to form a heterocycle Hy₉ and/or being optionally fused to a ring C¹⁰ of 4 to 7 atoms, these rings C⁹ and C¹⁰ optionally comprising a carbonyl or thiocarbonyl function and/or at least one substituent A₃ selected from OR₅, CF₃, a halogen and a C₁-C₂₀ alkyl radical,
4) one of the groups CF₃, CN, OR₄, SR₄, NR₄R′₄, NR₄C(═NR′₄)NR″₄R₄′, COR₄, CSR₄, COOR₄, CONR₄R′₄, NR₄COR′₄, NR₄CONR′₄R″₄, SO₂NR₄R′₄, NR₄SO₂R′₄, SO₂R₄, SiR₄R′₄R″₄, Si(OR₄)(OR′₄)OR″₄ and SO₃H, in which R₄, R′₄, R″₄ and R₄′ independently represent a hydrogen atom or a C₁-C₂₀ alkyl radical optionally substituted with a heterocycle Hy₁₀ or a group CONR₅R′₅;
e) A₂ is selected from:
1) a halogen atom,
2) one of the groups CF₃, CN, OR₄, SR₄, NR₄R′₄, OCOR₄, COR₄, CSR₄, COOR₄, SO₂R₄, SiR₄R′₄R″₄, NO₂ and OCF₃, in which R₄, R′₄ and R″₄ independently represent a hydrogen atom or a C₁-C₂₀ alkyl radical optionally substituted with a phenyl radical,
3) a C₁-C₂₀ alkyl radical optionally substituted with a group OR₅,
4) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function and/or at least one substituent A₃ selected from OR₅, CF₃, a halogen atom and a C₁-C₂₀ alkyl radical;
f) R₅ and R′₅ are independently selected from:
1) a hydrogen atom,
2) a C₁-C₂₀ alkyl radical;
g) Hy₁, Hy₂, Hy₄ to Hy₈, Hy₁₀ and Hy₁₁ independently represent a heterocycle of 4 to 7 atoms optionally having from 1 to 4 heteroatoms selected from among N, O and S and combinations thereof and/or comprising a carbonyl or thiocarbonyl function,
h) Hy₉ is a heterocycle of 4 to 15 atoms optionally having from 1 to 5 heteroatoms selected from among N, O and S and combinations thereof and/or comprising a carbonyl or thiocarbonyl function.

2. A regime or regimen for inhibiting type-1 15-hydroxyprostaglandin dehydrogenase, comprising administering to a mammalian organism in need of such treatment, for such period of time as required to elicit the desired response, a thus effective amount of at least one 2-oxyacetamide compound of formula (I) or a salt and/or solvate thereof: 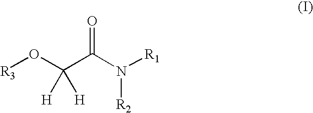
in which:
a) R₁ and R₂ are independently selected from:
1) a hydrogen atom, with R₁ different than R₂,
2) a C₁-C₂₀ alkyl radical optionally substituted with at least one substituent A₁,
3) a hydrocarbon-based ring C¹ of 3 to 7 atoms optionally fused to at least one ring C² of 4 to 7 atoms, the ring C² optionally containing at least one heteroatom to form a heterocycle Hy₂, these rings C¹ and C² optionally comprising a carbonyl or thiocarbonyl function and/or being substituted with at least one substituent A₂,
4) a heterocycle Hy₁ optionally fused to a ring C² of 4 to 7 atoms, the ring C² optionally containing at least one heteroatom to form a heterocycle Hy₂, these rings Hy₁ and C² optionally comprising a carbonyl or thiocarbonyl function and/or being substituted with at least one substituent A₁,
5) one of the groups C(═NR)R′, C(═NR)NR′R″, COR, CSR, COOR, CONRR′, SO₂R or SO₂NRR′;
b) R₃ is selected from:
1) a C₁-C₂₀ alkyl radical optionally substituted with at least one substituent A₁,
2) a hydrocarbon-based ring C³ of 3 to 7 atoms optionally fused to at least one ring C⁴ of 4 to 7 atoms optionally containing at least one heteroatom to form a heterocycle Hy₄, these rings C³ and C⁴ optionally comprising a carbonyl or thiocarbonyl function and/or being substituted with at least one substituent A₂,
3) a heterocycle Hy₃ selected from among pyrrole, furan, thiophene and pyrazole rings and optionally fused to a ring C⁵ constituting a phenyl, a pyridine or a pyrimidine, the heterocycle Hy₃ and the ring C⁵ being optionally substituted with at least one substituent A₁,
4) a heterocycle Hy₅ different than Hy₃ and optionally fused to a ring C⁶ of 4 to 7 atoms, these rings Hy₅ and C⁶ optionally comprising a carbonyl or thiocarbonyl function and/or being substituted with at least one substituent A₁, this ring C⁶ optionally containing at least one heteroatom to form a heterocycle Hy₆;
c) R, R′ and R″ are independently selected from:
1) a hydrogen atom,
2) a C₁-C₂₀ alkyl radical optionally substituted with at least one substituent A₁,
3) a ring C⁷ of 4 to 7 atoms optionally containing at least one heteroatom to form a heterocycle Hy₇ and/or being optionally fused to a ring C⁸ of 4 to 7 atoms optionally containing at least one heteroatom to form a heterocycle Hy₈, the rings C⁷ and C⁸ being optionally substituted with at least one substituent A₁ and optionally comprising a carbonyl or thiocarbonyl function;
d) A₁ is selected from:
1) a halogen atom,
2) a C₁-C₂₀ alkyl radical optionally substituted with a group OR₅,
3) a ring C⁹ of 4 to 15 atoms optionally containing at least one heteroatom to form a heterocycle Hy₉ and/or being optionally fused to a ring C¹⁰ of 4 to 7 atoms, these rings C⁹ and C¹⁰ optionally comprising a carbonyl or thiocarbonyl function and/or at least one substituent A₃ selected from OR₅, CF₃, a halogen and a C₁-C₂₀ alkyl radical,
4) one of the groups CF₃, CN, OR₄, SR₄, NR₄R′₄, NR₄C(═NR′₄)NR″₄R₄′, COR₄, CSR₄, COOR₄, CONR₄R′₄, NR₄COR′₄, NR₄CONR′₄R″₄, SO₂NR₄R′₄, NR₄SO₂R′₄, SO₂R₄, SiR₄R′₄R″₄, Si(OR₄)(OR′₄)OR″₄ and SO₃H, in which R₄, R′₄, R″₄ and R₄′ independently represent a hydrogen atom or a C₁-C₂₀ alkyl radical optionally substituted with a heterocycle Hy₁₀ or a group CONR₅R′₅;
e) A₂ is selected from:
1) a halogen atom,
2) one of the groups CF₃, CN, OR₄, SR₄, NR₄R′₄, OCOR₄, COR₄, CSR₄, COOR₄, SO₂R₄, SiR₄R′₄R″₄, NO₂ and OCF₃, in which R₄, R′₄ and R″₄ independently represent a hydrogen atom or a C₁-C₂₀ alkyl radical optionally substituted with a phenyl radical,
3) a C₁-C₂₀ alkyl radical optionally substituted with a group OR₅,
4) a ring C¹¹ of 4 to 7 atoms optionally containing at least one heteroatom to form a heterocycle Hy₁₁, and/or being optionally fused to a ring C¹² of 4 to 7 atoms, these rings C¹¹ and C¹² optionally comprising a carbonyl or thiocarbonyl function and/or at least one substituent A₃ selected from OR₅, CF₃, a halogen atom and a C₁-C₂₀ alkyl radical;
f) R₅ and R′₅ are independently selected from:
1) a hydrogen atom,
2) a C₁-C₂₀ alkyl radical;
g) Hy₁, Hy₂, Hy₄ to Hy₈, Hy₁₀ and Hy₁₁ independently represent a heterocycle of 4 to 7 atoms optionally having from 1 to 4 heteroatoms selected from among N, O and S and combinations thereof and/or comprising a carbonyl or thiocarbonyl function,
h) Hy₉ is a heterocycle of 4 to 15 atoms optionally having from 1 to 5 heteroatoms selected from among N, O and S and combinations thereof and/or comprising a carbonyl or thiocarbonyl function.

3. The regime or regimen as defined by claim 2, for inhibiting the human form of said type-1 15-hydroxyprostaglandin dehydrogenase.

4. A regime or regimen for treating a human disorder associated with the human form of type-1 15-hydroxyprostaglandin dehydrogenase, comprising administering to an individual in need of such treatment, for such period of time as required to elicit the desired response, a thus effective amount of at least one 2-oxyacetamide compound of formula (I) or a salt and/or solvate thereof: 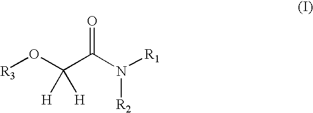
in which:
a) R₁ and R₂ are independently selected from:
1) a hydrogen atom, with R₁ different than R₂,
2) a C₁-C₂₀ alkyl radical optionally substituted with at least one substituent A₁,
3) a hydrocarbon-based ring C¹ of 3 to 7 atoms optionally fused to at least one ring C² of 4 to 7 atoms, the ring C² optionally containing at least one heteroatom to form a heterocycle Hy₂, these rings C¹ and C² optionally comprising a carbonyl or thiocarbonyl function and/or being substituted with at least one substituent A₂,
4) a heterocycle Hy₁ optionally fused to a ring C² of 4 to 7 atoms, the ring C² optionally containing at least one heteroatom to form a heterocycle Hy₂, these rings Hy₁ and C² optionally comprising a carbonyl or thiocarbonyl function and/or being substituted with at least one substituent A₁,
5) one of the groups C(═NR)R′, C(═NR)NR′R″, COR, CSR, COOR, CONRR′, SO₂R or SO₂NRR′;
b) R₃ is selected from:
1) a C₁-C₂₀ alkyl radical optionally substituted with at least one substituent A₁,
2) a hydrocarbon-based ring C³ of 3 to 7 atoms optionally fused to at least one ring C⁴ of 4 to 7 atoms optionally containing at least one heteroatom to form a heterocycle Hy₄, these rings C³ and C⁴ optionally comprising a carbonyl or thiocarbonyl function and/or being substituted with at least one substituent A₂,
3) a heterocycle Hy₃ selected from among pyrrole, furan, thiophene and pyrazole rings and optionally fused to a ring C⁵ constituting a phenyl, a pyridine or a pyrimidine, the heterocycle Hy₃ and the ring C⁵ being optionally substituted with at least one substituent A₁,
4) a heterocycle Hy₅ different than Hy₃ and optionally fused to a ring C⁶ of 4 to 7 atoms, these rings Hy₅ and C⁶ optionally comprising a carbonyl or thiocarbonyl function and/or being substituted with at least one substituent A₁, this ring C⁶ optionally containing at least one heteroatom to form a heterocycle Hy₆;
c) R, R′ and R″ are independently selected from:
1) a hydrogen atom,
2) a C₁-C₂₀ alkyl radical optionally substituted with at least one substituent A₁,
3) a ring C⁷ of 4 to 7 atoms optionally containing at least one heteroatom to form a heterocycle Hy₇ and/or being optionally fused to a ring C⁸ of 4 to 7 atoms optionally containing at least one heteroatom to form a heterocycle Hy₈, the rings C⁷ and C⁸ being optionally substituted with at least one substituent A₁ and optionally comprising a carbonyl or thiocarbonyl function;
d) A₁ is selected from:
1) a halogen atom,
2) a C₁-C₂₀ alkyl radical optionally substituted with a group OR₅,
3) a ring C⁹ of 4 to 15 atoms optionally containing at least one heteroatom to form a heterocycle Hy₉ and/or being optionally fused to a ring C¹⁰ of 4 to 7 atoms, these rings C⁹ and C¹⁰ optionally comprising a carbonyl or thiocarbonyl function and/or at least one substituent A₃ selected from OR₅, CF₃, a halogen and a C₁-C₂₀ alkyl radical,
4) one of the groups CF₃, CN, OR₄, SR₄, NR₄R′₄, NR₄C(═NR′₄)NR″₄R₄′, COR₄, CSR₄, COOR₄, CONR₄R′₄, NR₄COR′₄, NR₄CONR′₄R″₄, SO₂NR₄R′₄, NR₄SO₂R′₄, SO₂R₄, SiR₄R′₄R″₄, Si(OR₄)(OR′₄)OR″₄ and SO₃H, in which R₄, R′₄, R″₄ and R₄′ independently represent a hydrogen atom or a C₁-C₂₀ alkyl radical optionally substituted with a heterocycle Hy₁₀ or a group CONR₅R′₅;
e) A₂ is selected from:
1) a halogen atom,
2) one of the groups CF₃, CN, OR₄, SR₄, NR₄R′₄, OCOR₄, COR₄, CSR₄, COOR₄, SO₂R₄, SiR₄R′₄R″₄, NO₂ and OCF₃, in which R₄, R′₄ and R″₄ independently represent a hydrogen atom or a C₁-C₂₀ alkyl radical optionally substituted with a phenyl radical,
3) a C₁-C₂₀ alkyl radical optionally substituted with a group OR₅,
4) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function and/or at least one substituent A₃ selected from OR₅, CF₃, a halogen atom and a C₁-C₂₀ alkyl radical;
f) R₅ and R′₅ are independently selected from:
1) a hydrogen atom,
2) a C₁-C₂₀ alkyl radical;
g) Hy₁, Hy₂, Hy₄ to Hy₈, Hy₁₀ and Hy₁₁ independently represent a heterocycle of 4 to 7 atoms optionally having from 1 to 4 heteroatoms selected from among N, O and S and combinations thereof and/or comprising a carbonyl or thiocarbonyl function,
h) Hy₉ is a heterocycle of 4 to 15 atoms optionally having from 1 to 5 heteroatoms selected from among N, O and S and combinations thereof and/or comprising a carbonyl or thiocarbonyl function.

5. The regime or regimen as defined by claim 1, for inducing and/or stimulating the growth of human keratin fibers which comprise head hair, the eyebrows, the eyelashes, beard hair, moustache hair or pubic hair and/or for retarding the loss and/or increasing the density thereof and/or for caring for and/or making up such human keratin fibers.

6. A regime or regimen for treating androgenic alopecia and/or alopecia of natural origin, comprising administering to an individual in need of such treatment, for such period of time as required to elicit the desired response, a thus effective amount of at least one 2-oxyacetamide compound of formula (I) or a salt and/or solvate thereof: 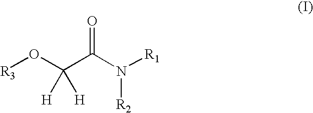
in which:
a) R₁ and R₂ are independently selected from:
1) a hydrogen atom, with R₁ different than R₂,
2) a C₁-C₂₀ alkyl radical optionally substituted with at least one substituent A₁,
3) a hydrocarbon-based ring C¹ of 3 to 7 atoms optionally fused to at least one ring C² of 4 to 7 atoms, the ring C² optionally containing at least one heteroatom to form a heterocycle Hy₂, these rings C¹ and C² optionally comprising a carbonyl or thiocarbonyl function and/or being substituted with at least one substituent A₂,
4) a heterocycle Hy₁ optionally fused to a ring C² of 4 to 7 atoms, the ring C² optionally containing at least one heteroatom to form a heterocycle Hy₂, these rings Hy₁, and C² optionally comprising a carbonyl or thiocarbonyl function and/or being substituted with at least one substituent A₁,
5) one of the groups C(═NR)R′, C(═NR)NR′R″, COR, CSR, COOR, CONRR′, SO₂R or SO₂NRR′;
b) R₃ is selected from:
1) a C₁-C₂₀ alkyl radical optionally substituted with at least one substituent A₁,
2) a hydrocarbon-based ring C³ of 3 to 7 atoms optionally fused to at least one ring C⁴ of 4 to 7 atoms optionally containing at least one heteroatom to form a heterocycle Hy₄, these rings C³ and C⁴ optionally comprising a carbonyl or thiocarbonyl function and/or being substituted with at least one substituent A₂,
3) a heterocycle Hy₃ selected from among pyrrole, furan, thiophene and pyrazole rings and optionally fused to a ring C⁵ constituting a phenyl, a pyridine or a pyrimidine, the heterocycle Hy₃ and the ring C⁵ being optionally substituted with at least one substituent A₁,
4) a heterocycle Hy₅ different than Hy₃ and optionally fused to a ring C⁶ of 4 to 7 atoms, these rings Hy₅ and C⁶ optionally comprising a carbonyl or thiocarbonyl function and/or being substituted with at least one substituent A₁, this ring C⁶ optionally containing at least one heteroatom to form a heterocycle Hy₆;
c) R, R′ and R″ are independently selected from:
1) a hydrogen atom,
2) a C₁-C₂₀ alkyl radical optionally substituted with at least one substituent A₁,
3) a ring C⁷ of 4 to 7 atoms optionally containing at least one heteroatom to form a heterocycle Hy₇ and/or being optionally fused to a ring C⁸ of 4 to 7 atoms optionally containing at least one heteroatom to form a heterocycle Hy₈, the rings C⁷ and C⁸ being optionally substituted with at least one substituent A₁ and optionally comprising a carbonyl or thiocarbonyl function;
d) A₁ is selected from:
1) a halogen atom,
2) a C₁-C₂₀ alkyl radical optionally substituted with a group OR₅,
3) a ring C⁹ of 4 to 15 atoms optionally containing at least one heteroatom to form a heterocycle Hy₉ and/or being optionally fused to a ring C¹⁰ of 4 to 7 atoms, these rings C⁹ and C¹⁰ optionally comprising a carbonyl or thiocarbonyl function and/or at least one substituent A₃ selected from OR₅, CF₃, a halogen and a C₁-C₂₀ alkyl radical,
4) one of the groups CF₃, CN, OR₄, SR₄, NR₄R′₄, NR₄C(═NR′₄)NR″₄R₄′, COR₄, CSR₄, COOR₄, CONR₄R′₄, NR₄COR′₄, NR₄CONR′₄R″₄, SO₂NR₄R′₄, NR₄SO₂R′₄, SO₂R₄, SiR₄R′₄R″₄, Si(OR₄)(OR′₄)OR″₄ and SO₃H, in which R₄, R′₄, R″₄ and R₄′ independently represent a hydrogen atom or a C₁-C₂₀ alkyl radical optionally substituted with a heterocycle Hy₁₀ or a group CONR₅R′₅;
e) A₂ is selected from:
1) a halogen atom,
2) one of the groups CF₃, CN, OR₄, SR₄, NR₄R′₄, OCOR₄, COR₄, CSR₄, COOR₄, SO₂R₄, SiR₄R′₄R″₄, NO₂ and OCF₃, in which R₄, R′₄ and R″₄ independently represent a hydrogen atom or a C₁-C₂₀ alkyl radical optionally substituted with a phenyl radical,
3) a C₁-C₂₀ alkyl radical optionally substituted with a group OR₅,
4) a ring C¹¹ of 4 to 7 atoms optionally containing at least one heteroatom to form a heterocycle Hy₁₁, and/or being optionally fused to a ring C¹² of 4 to 7 atoms, these rings C¹¹ and C¹² optionally comprising a carbonyl or thiocarbonyl function and/or at least one substituent A₃ selected from OR₅, CF₃, a halogen atom and a C₁-C₂₀ alkyl radical;
f) R₅ and R′₅ are independently selected from:
1) a hydrogen atom,
2) a C₁-C₂₀ alkyl radical;
g) Hy₁, Hy₂, Hy₄ to Hy₈, Hy₁₀ and Hy₁₁ independently represent a heterocycle of 4 to 7 atoms optionally having from 1 to 4 heteroatoms selected from among N, O and S and combinations thereof and/or comprising a carbonyl or thiocarbonyl function,
h) Hy₉ is a heterocycle of 4 to 15 atoms optionally having from 1 to 5 heteroatoms selected from among N, O and S and combinations thereof and/or comprising a carbonyl or thiocarbonyl function.

7. The regime or regimen as defined by claim 6, comprising topically applying said at least one 2-oxyacetamide compound of formula (I) or salt and/or solvate thereof onto the affected area of the skin and/or keratinous fibers of such individual.

8. The regime or regimen as defined by claims 1 or 2, wherein formula (1):
R₁ is hydrogen and R₂ is one of the following groups:
a saturated C₁-C₁₀ alkyl radical, optionally substituted with one or two substituents A₁,
a saturated hydrocarbon-based ring of 3 to 6 carbon atoms,
a saturated or unsaturated heterocycle Hy₁ of 5, 6 or 7 atoms, comprising 1 or 2 heteroatoms selected from among O, N and S and optionally a carbonyl function and/or from 1 to 4 substituents A′₂,
a phenyl ring substituted with one or two substituents A″₂ selected from among halogen atom, NO₂, OCF₃, CF₃, OR₄, OCH₂R₄, COOR₄, a C₁-C₁₀ alkyl radical, and a hydrocarbon-based or heterocyclic aromatic ring,
a phenyl ring fused to one or two hydrocarbon-based or heterocyclic rings of 5 to 6 atoms.

9. The regime or regimen as defined by claim 1, wherein formula (I) at least one of R₁ and R₂ is an Hy₁ heterocycle selected from the group consisting of azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, dihydrothiazole, thiazolidine, dihydropyrazole, pyrazolidine, oxazole, dihydrooxazole, oxazolidine, isoxazole, dihydroisoxazole isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine.

10. The regime or regimen as defined by claim 1, wherein formula (I):
R₃ is one of the following groups:
a hydrocarbon-based ring, optionally substituted with one or more substituents A₂′ selected from among a phenyl radical, COOR₄, OR₄, COR₄, CN, a heterocycle containing 5 or 6 atoms and comprising 1 or 2 heteroatoms selected from among O, N and S, a halogen atom, a phenyl radical optionally substituted with CN, a linear or branched C₁-C₁₀ alkyl radical in which R₄ is a hydrogen atom or a C₁-C₁₀ alkyl radical,
a phenyl ring fused to one or two hydrocarbon-based or heterocyclic rings C⁴ of 5 to 6 atoms, the ring(s) C⁴ optionally comprising a carbonyl function and/or optionally being substituted with a saturated C₁-C₁₀ alkyl radical, this phenyl and this or these other ring(s) C⁴ thus forming fused rings,
a saturated C₁-C₁₀ alkyl radical, optionally substituted with one or two substituents A₁.

11. The regime or regimen as defined by claim 1, wherein formula (I), the heterocycles Hy₂, Hy₄, Hy₆, Hy₇, Hy₈, Hy₉, Hy₁₀ and Hy₁₁ are independently selected from the group consisting of azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, thiazole, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine rings and a 15-C-5 crown ether for Hy₉.

12. The regime or regimen as defined by claim 11, wherein formula (I), the heterocycles Hy₂, Hy₄, Hy₆, Hy₇, Hy₈, Hy₉, Hy₁₀ and Hy₁₁ are independently selected from the group consisting of pyrrole, pyrrolidine, imidazole, furan, thiophene, oxazole, thiazole, isoxazole, isothiazole, oxadiazole, pyrazole, tetrazole, pyridine, pyrimidine, triazole, pyrazine, pyridazine, piperidine, piperazine or morpholine rings and a 15-C-5 crown ether for Hy₉.

13. The regime or regimen as defined by claim 1, wherein formula (I), A₁ is a hydrocarbon-based ring or a heterocycle comprising 1 or 2 heteroatoms selected from among O and N, this hydrocarbon-based or heterocyclic ring comprising 5 to 6 atoms and optionally a carbonyl function and/or a substituent A₃; or a group selected from among SiR₄R′₄R″₄, COOR₄, NR₄R′₄, OR₄, SR₄ and CONR₄R′₄.

14. The regime or regimen as defined by claim 1, wherein formula (I), A₃ is a C₁-C₁₀ alkyl radical, CF₃, a halogen atom, OH or OCH₃.

15. The regime or regimen as defined by claim 1, wherein formula (I), Hy₁ is a γ-butyrolactone, a piperidine or a 1,3-benzodioxole optionally substituted with a C₁-C₄ alkyl radical.

16. The regime or regimen as defined by claim 1, said at least one 2-oxyacetamide compound of formula (I) comprising a salt thereof selected from the group consisting of sodium or potassium salts, the ammonium, zinc (Zn²⁺), calcium (Ca²⁺), copper (Cu²⁺), iron (Fe²⁺ and Fe³⁺), strontium (Sr²⁺), magnesium (Mg²⁺) and manganese (Mn²⁺) salts, triethanolamine, monoethanolamine, ethanolamine, hexadecylamine and N,N,N′,N′-tetrakis(2-hydroxypropyl)ethylene diamine salts, hydroxides, hydrohalides, carbonates, hydrogen carbonates, citrates, lactates, glycolates, gluconates, acetates, propionates, fumarates, oxalates, tartrates, sulfates, phosphates and hydrogen phosphates.

17. The regime or regimen as defined by claim 1, said at least one 2-oxyacetamide compound of formula (I) comprising a hydrate thereof.

18. The regime or regimen as defined by claim 1, said at least one 2-oxyacetamide compound having the formula (1a) or a salt and/or solvate thereof: 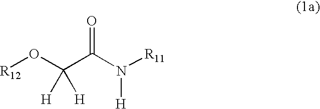
in which:
α) R₁₁, is selected from:
1) a C₁-C₁₀ alkyl radical optionally substituted with at least one substituent A⁴,
2) a hydrocarbon-based ring C¹⁵ of 5 to 6 atoms optionally substituted with at least one substituent A₅;
β) R₁₂ is selected from:
1) a C₁-C₁₀ alkyl radical optionally substituted with at least one substituent A₄,
2) a hydrocarbon-based ring C¹⁶ of 5 to 6 atoms, optionally fused to at least one ring C¹⁷ of 5 to 6 atoms optionally containing at least one heteroatom to form a heterocycle Hy₁₇, these rings C¹⁶ and C¹⁷ optionally comprising a carbonyl function and/or being substituted with at least one substituent A₅;
γ) A₄ is selected from:
1) a ring C¹⁸ of 5 to 6 atoms optionally containing at least one heteroatom to form a heterocycle Hy₁₈, this ring or this heterocycle being optionally substituted with at least one substituent A₆ selected from among OR₁₄, CF₃, a halogen atom, and a C₁-C₁₀ alkyl radical;
2) one of the groups CF₃, CN, OR₁₃, SR₁₃, NR₁₃R′₁₃, NR₁₃C(═NR′₁₃)NR″₁₃R′₁₃, COR₁₃, COOR₁₃, CONR₁₃R′₁₃, NR₁₃COR′₁₃, NR₁₃CONR′₁₃R″₁₃, SO₂NR₁₃R′₁₃, NR₁₃SO₂R′₁₃, SO₂R₁₃, SiR₁₃R′₁₃R″₁₃, and SO₃H, in which R₁₃, R′₁₃, R″₁₃ and R₁₃′ independently represent a hydrogen atom or a C₁-C₁₀ alkyl radical;
δ) A₅ is selected from:
1) a halogen atom,
2) one of the groups CF₃, CN, OR₁₅, SR₁₅, NR₁₅R′₁₅, OCOR₁₅, COR₁₅, COOR₁₅, SO₂R₁₅, SiR₁₅R′₁₅R″₁₅, NO₂ and OCF₃, in which R₁₅, R′₁₅ and R″₁₅ independently represent a hydrogen atom or a C₁-C₁₀ alkyl radical optionally substituted with a phenyl radical,
3) a C₁-C₁₀ alkyl radical optionally substituted with a group OR₁₄,
4) a ring C¹⁹ of 5 to 6 atoms optionally containing at least one heteroatom to form a heterocycle Hy₁₉ and/or optionally comprising a carbonyl function;
ε) R₁₄ is selected from:
1) a hydrogen atom,
2) a C₁-C₁₀ alkyl radical;
η) Hy₁₇, Hy₁₈ and Hy₁₉ independently represent a heterocycle of 5 to 6 atoms optionally having from 1 to 4 heteroatoms selected from among N, O and S and combinations thereof and/or comprising a carbonyl function.

19. The regime or regimen as defined by claim 1, said at least one 2-oxyacetamide compound having one of the following formulae (II) to (V) or a salt and/or solvate thereof:
Formula (II): 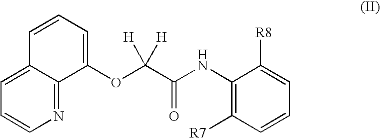
Formula (III): 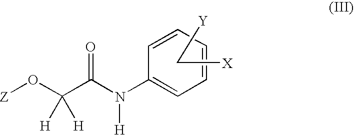
Formula (IV): 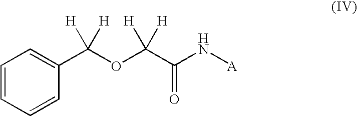
Formula (V): 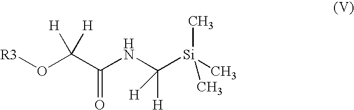
in which:
(i) R7 and R8 independently represent:
1) a hydrogen atom,
2) a halogen atom F or Cl,
3) one of the groups CF₃, CN, OR₄, SR₄, NHR₆, NR₆R′₆, OCOR₄, COR₄, CSR₄, COOR₄, SO₂R₄, SiR₄R′₄R″₄ and OCF₃, in which R₄, R′₄ and R″₄ independently represent a hydrogen atom or a C₁-C₂₀ alkyl radical optionally substituted with a phenyl radical and in which R₆ and R′₆ represent a C₁-C₂₀ alkyl radical optionally substituted with a phenyl radical,
4) a C₁-C₂₀ alkyl radical optionally substituted with a group OR₅ in which R₅ is a hydrogen atom or a C₁-C₂₀ alkyl radical,
5) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function;
(ii) Z is one of the following rings and heterocycles: 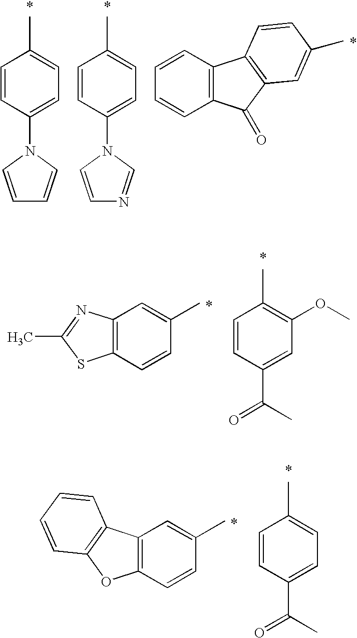
(iii) X and Y independently represent a hydrogen atomor A₂ or may form a fused ring C¹³ of 4 to 7 atoms, this ring C¹³ optionally comprising at least one heteroatom, being optionally substituted with at least one substituent A₂, and optionally fused to another ring C¹⁴;
(iv) A is one of the following three groups: 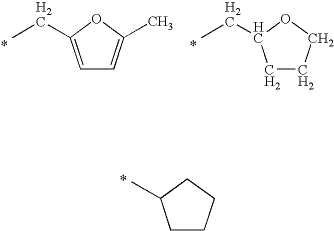
(v) R₃ is as defined for formula (I); with the proviso that when Z is the dibenzofuran heterocycle, X and Y independently represent:
1) a hydrogen atom,
2) a halogen atom,
3) one of the groups CF₃, CN, OR₉, SR₄, NR₄R′₄, OCOR₄, COR₄, CSR₄, COOR₄, SO₂R₄, SiR₄R′₄R″₄, NO₂ and OCF₃, in which R₄, R′₄ and R″₄ independently represent a hydrogen atom or a C₁-C₂₀ alkyl radical optionally substituted with a phenyl radical and in which R₉ is a hydrogen atom or a C₂-C₂₀ alkyl radical optionally substituted with a phenyl radical,
4) a C₂-C₂₀ alkyl radical optionally substituted with a group OR₅,
5) a ring C¹¹ of 4 to 7 atoms optionally containing at least one heteroatom to form a heterocycle Hy₁₁, and/or being optionally fused to a ring C¹² of 4 to 7 atoms, these rings C¹¹ and C¹² optionally comprising a carbonyl or thiocarbonyl function; or with the proviso that when Z is the ring: 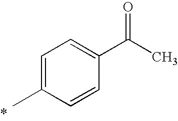
X and Y independently represent:
1) a hydrogen atom, in this case X is different than Y,
2) fluorine,
3) one of the groups OR₁₀, SR₄, NR₄R′₄, OCOR₄, COR₄, CSR₄, COOR₆, SiR₄R′₄R″₄ and OCF₃, in which R₄, R′₄ and R″₄ independently represent a hydrogen atom or a C₁-C₂₀ alkyl radical optionally substituted with a phenyl radical, in which R₆ is a C₁-C₂₀ alkyl radical optionally substituted with a phenyl radical and in which R₁₀ is a hydrogen atom or a benzyl radical or a C₃-C₂₀ alkyl radical optionally substituted with a phenyl radical,
4) a C₂-C₂₀ alkyl radical optionally substituted with a group OR₅,
5) with the proviso that X and Y may also form a 5-atom ring C¹³ optionally containing one or two oxygen atoms and being optionally fused to another ring C¹⁴.

20. The regime or regimen as defined by claim 1, said at least one 2-oxyacetamide compound having one of the following formulae or a salt and/or solvate thereof:

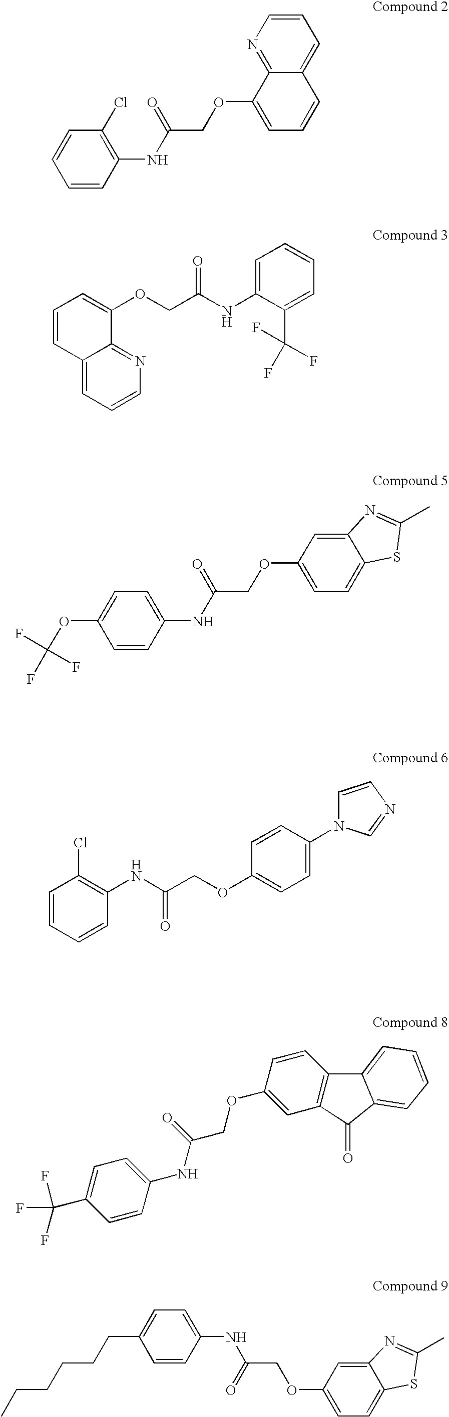

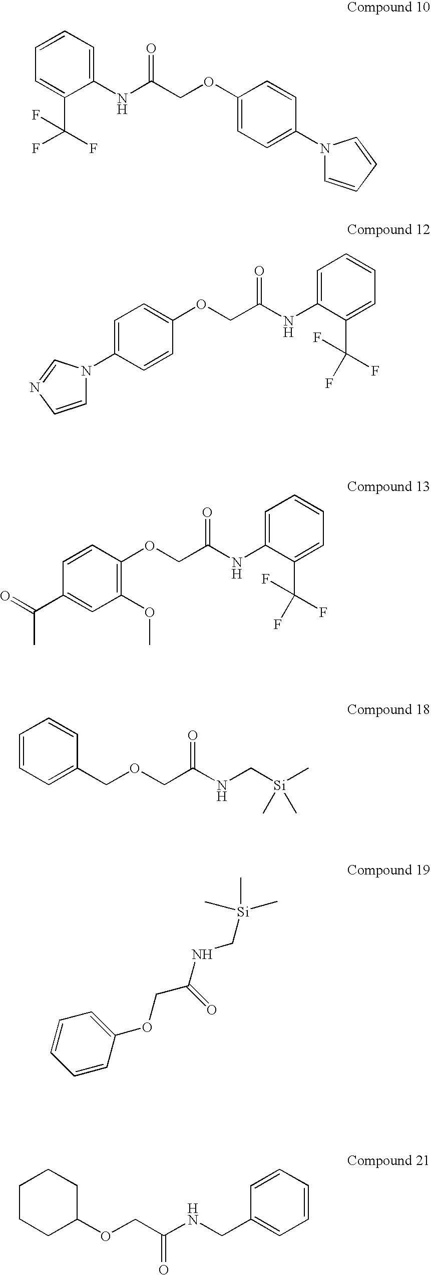

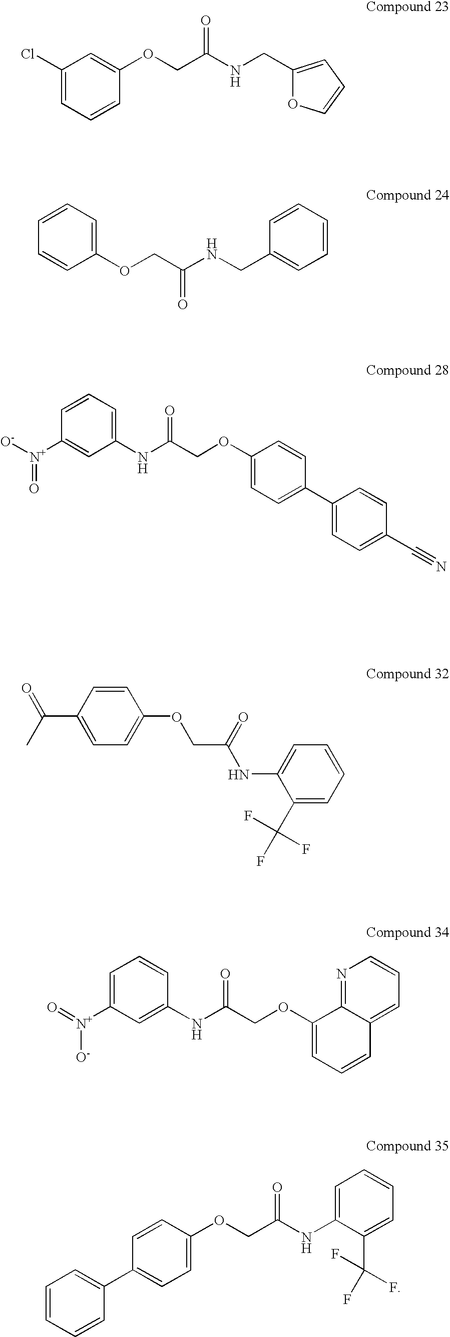

21. A topically applicable cosmetic/pharmaceutical composition formulated as a hair cream or hair lotion, a shampoo, a conditioner, a mascara for the hair or for the eyelashes, or an aqueous, alcoholic or aqueous-alcoholic solution or suspension, useful for inducing and/or stimulating the growth of human keratin fibers and/or for retarding the loss and/or increasing the density thereof and/or for caring for and/or making up such keratin fibers, comprising a thus effective amount of at least one 2-oxyacetamide compound of formula (I) or a salt and/or solvate thereof: 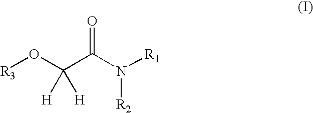
in which:
a) R₁ and R₂ are independently selected from:
1) a hydrogen atom, with R₁ different than R₂,
2) a C₁-C₂₀ alkyl radical optionally substituted with at least one substituent A₁,
3) a hydrocarbon-based ring C¹ of 3 to 7 atoms optionally fused to at least one ring C² of 4 to 7 atoms, the ring C² optionally containing at least one heteroatom to form a heterocycle Hy₂, these rings C¹ and C² optionally comprising a carbonyl or thiocarbonyl function and/or being substituted with at least one substituent A₂,
4) a heterocycle Hy₁, optionally fused to a ring C² of 4 to 7 atoms, the ring C² optionally containing at least one heteroatom to form a heterocycle Hy₂, these rings Hy₁ and C² optionally comprising a carbonyl or thiocarbonyl function and/or being substituted with at least one substituent A₁,
5) one of the groups C(═NR)R′, C(═NR)NR′R″, COR, CSR, COOR, CONRR′, SO₂R or SO₂NRR′;
b) R₃ is selected from:
1) a C₁-C₂₀ alkyl radical optionally substituted with at least one substituent A₁,
2) a hydrocarbon-based ring C³ of 3 to 7 atoms optionally fused to at least one ring C⁴ of 4 to 7 atoms optionally containing at least one heteroatom to form a heterocycle Hy₄, these rings C³ and C⁴ optionally comprising a carbonyl or thiocarbonyl function and/or being substituted with at least one substituent A₂,
3) a heterocycle Hy₃ selected from among pyrrole, furan, thiophene and pyrazole rings and optionally fused to a ring C⁵ constituting a phenyl, a pyridine or a pyrimidine, the heterocycle Hy₃ and the ring C⁵ being optionally substituted with at least one substituent A₁,
4) a heterocycle Hy₅ different than Hy₃ and optionally fused to a ring C⁶ of 4 to 7 atoms, these rings Hy₅ and C⁶ optionally comprising a carbonyl or thiocarbonyl function and/or being substituted with at least one substituent A₁, this ring C⁶ optionally containing at least one heteroatom to form a heterocycle Hy₆;
c) R, R′ and R″ are independently selected from:
1) a hydrogen atom,
2) a C₁-C₂₀ alkyl radical optionally substituted with at least one substituent A₁,
3) a ring C⁷ of 4 to 7 atoms optionally containing at least one heteroatom to form a heterocycle Hy₇ and/or being optionally fused to a ring C⁸ of 4 to 7 atoms optionally containing at least one heteroatom to form a heterocycle Hy₈, the rings C⁷ and C⁸ being optionally substituted with at least one substituent A₁ and optionally comprising a carbonyl or thiocarbonyl function;
d) A₁ is selected from:
1) a halogen atom,
2) a C₁-C₂₀ alkyl radical optionally substituted with a group OR₅,
3) a ring C⁹ of 4 to 15 atoms optionally containing at least one heteroatom to form a heterocycle Hy₉ and/or being optionally fused to a ring C¹⁰ of 4 to 7 atoms, these rings C⁹ and C¹⁰ optionally comprising a carbonyl or thiocarbonyl function and/or at least one substituent A₃ selected from OR₅, CF₃, a halogen and a C₁-C₂₀ alkyl radical,
4) one of the groups CF₃, CN, OR₄, SR₄, NR₄R′₄, NR₄C(═NR′₄)NR″₄R₄′, COR₄, CSR₄, COOR₄, CONR₄R′₄, NR₄COR′₄, NR₄CONR′₄R″₄, SO₂NR₄R′₄, NR₄SO₂R′₄, SO₂R₄, SiR₄R′₄R″₄, Si(OR₄)(OR′₄)OR″₄ and SO₃H, in which R₄, R′₄, R″₄ and R₄′ independently represent a hydrogen atom or a C₁-C₂₀ alkyl radical optionally substituted with a heterocycle Hy₁₀ or a group CONR₅R′₅;
e) A₂ is selected from:
1) a halogen atom,
2) one of the groups CF₃, CN, OR₄, SR₄, NR₄R′₄, OCOR₄, COR₄, CSR₄, COOR₄, SO₂R₄, SiR₄R′₄R″₄, NO₂ and OCF₃, in which R₄, R′₄ and R″₄ independently represent a hydrogen atom or a C₁-C₂₀ alkyl radical optionally substituted with a phenyl radical,
3) a C₁-C₂₀ alkyl radical optionally substituted with a group OR₅,
4) a ring C¹¹ of 4 to 7 atoms optionally containing at least one heteroatom to form a heterocycle Hy₁₁, and/or being optionally fused to a ring C¹² of 4 to 7 atoms, these rings C¹¹ and C¹² optionally comprising a carbonyl or thiocarbonyl function and/or at least one substituent A₃ selected from OR₅, CF₃, a halogen atom and a C₁-C₂₀ alkyl radical;
f) R₅ and R′₅ are independently selected from:
1) a hydrogen atom,
2) a C₁-C₂₀ alkyl radical;
g) Hy₁Hy₂, Hy₄ to Hy₈, Hy₁₀ and Hy₁₁ independently represent a heterocycle of 4 to 7 atoms optionally having from 1 to 4 heteroatoms selected from among N, O and S and combinations thereof and/or comprising a carbonyl or thiocarbonyl function,
h) Hy₉ is a heterocycle of 4 to 15 atoms optionally having from 1 to 5 heteroatoms selected from among N, O and S and combinations thereof and/or comprising a carbonyl or thiocarbonyl function, formulated into a topically applicable physiologically acceptable medium therefor.

22. The topically applicable cosmetic/pharmaceutical composition as defined by claim 21, said at least one 2-oxyacetamide compound of formula (I) or a salt or solvate thereof comprising from 10⁻² to 10% by weight thereof.

23. The topically applicable cosmetic/pharmaceutical composition as defined by claim 21, said at least one 2-oxyacetamide compound of formula (I) or a salt or solvate thereof comprising from 10⁻² to 2% by weight thereof.

24. The topically applicable cosmetic/pharmaceutical composition as defined by claim 21, wherein formula (I):
R₁ is hydrogen and R₂ is one of the following groups:
a saturated C₁-C₁₀ alkyl radical, optionally substituted with one or two substituents A₁,
a saturated hydrocarbon-based ring of 3 to 6 carbon atoms,
a saturated or unsaturated heterocycle Hy₁ of 5, 6 or 7 atoms, comprising 1 or 2 heteroatoms selected from among O, N and S and optionally a carbonyl function and/or from 1 to 4 substituents A′₂,
a phenyl ring substituted with one or two substituents A″₂ selected from among halogen atom, NO₂, OCF₃, CF₃, OR₄, OCH₂R₄, COOR₄, a C₁-C₁₀ alkyl radical, and a hydrocarbon-based or heterocyclic aromatic ring,
a phenyl ring fused to one or two hydrocarbon-based or heterocyclic rings of 5 to 6 atoms.

25. The topically applicable cosmetic/pharmaceutical composition as defined by claim 21, wherein formula (I) at least one of R₁ and R₂ is an Hy1 heterocycle selected from the group consisting of azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, dihydrothiazole, thiazolidine, dihydropyrazole, pyrazolidine, oxazole, dihydrooxazole, oxazolidine, isoxazole, dihydroisoxazole isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine.

26. The topically applicable cosmetic/pharmaceutical composition as defined by claim 21, wherein formula (I):
R₃ is one of the following groups:
a hydrocarbon-based ring, optionally substituted with one or more substituents A₂′ selected from among a phenyl radical, COOR₄, OR₄, COR₄, CN, a heterocycle containing 5 or 6 atoms and comprising 1 or 2 heteroatoms selected from among O, N and S, a halogen atom, a phenyl radical optionally substituted with CN, a linear or branched C₁-C₁₀ alkyl radical in which R₄ is a hydrogen atom or a C₁-C₁₀ alkyl radical,
a phenyl ring fused to one or two hydrocarbon-based or heterocyclic rings C⁴ of 5 to 6 atoms, the ring(s) C⁴ optionally comprising a carbonyl function and/or optionally being substituted with a saturated C₁-C₁₀ alkyl radical, this phenyl and this or these other ring(s) C⁴ thus forming fused rings,
a saturated C₁-C₁₀ alkyl radical, optionally substituted with one or two substituents A₁.

27. The topically applicable cosmetic/pharmaceutical composition as defined by claim 21, wherein formula (I), the heterocycles Hy₂, Hy₄, Hy₆, Hy₇, Hy₈, Hy₉, Hy₁₀ and Hy₁₁ are independently selected from the group consisting of azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, thiazole, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine rings and a 15-C-5 crown ether for Hy₉.

28. The topically applicable cosmetic/pharmaceutical composition as defined by claim 27, wherein formula (I), the heterocycles Hy₂, Hy₄, Hy₆, Hy₇, Hy₈, Hy₉, Hy₁₀ and Hy₁₁ are independently selected from the group consisting of pyrrole, pyrrolidine, imidazole, furan, thiophene, oxazole, thiazole, isoxazole, isothiazole, oxadiazole, pyrazole, tetrazole, pyridine, pyrimidine, triazole, pyrazine, pyridazine, piperidine, piperazine or morpholine rings and a 15-C-5 crown ether for Hy₉.

29. The topically applicable cosmetic/pharmaceutical composition as defined by claim 21, wherein formula (I), A₁ is a hydrocarbon-based ring or a heterocycle comprising 1 or 2 heteroatoms selected from among O and N, this hydrocarbon-based or heterocyclic ring comprising 5 to 6 atoms and optionally a carbonyl function and/or a substituent A₃; or a group selected from among SiR₄R′₄R″₄, COOR₄, NR₄R′₄, OR₄, SR₄ and CONR₄R′₄.

30. The topically applicable cosmetic/pharmaceutical composition as defined by claim 21, wherein formula (I), A₃ is a C₁-C₁₀ alkyl radical, CF₃, a halogen atom, OH or OCH₃.

31. The topically applicable cosmetic/pharmaceutical composition as defined by claim 21, wherein formula (I), Hy₁, is a γ-butyrolactone, a piperidine or a 1,3-benzodioxole optionally substituted with a C₁-C₄ alkyl radical.

32. The topically applicable cosmetic/pharmaceutical composition as defined by claim 21, said at least one 2-oxyacetamide compound of formula (I) comprising a salt thereof selected from the group consisting of sodium or potassium salts, the ammonium, zinc (Zn²⁺), calcium (Ca²⁺), copper (Cu²⁺), iron (Fe²⁺ and Fe³⁺), strontium (Sr²⁺), magnesium (Mg²⁺) and manganese (Mn²⁺) salts, triethanolamine, monoethanolamine, ethanolamine, hexadecylamine and N,N,N′,N′-tetrakis(2-hydroxypropyl)ethylene diamine salts, hydroxides, hydrohalides, carbonates, hydrogen carbonates, citrates, lactates, glycolates, gluconates, acetates, propionates, fumarates, oxalates, tartrates, sulfates, phosphates and hydrogen phosphates.

33. The topically applicable cosmetic/pharmaceutical composition as defined by claim 21, said at least one 2-oxyacetamide compound of formula (I) comprising a hydrate thereof.

34. The topically applicable cosmetic/pharmaceutical composition as defined by claim 21, said at least one 2-oxyacetamide compound having the formula (1a) or a salt and/or solvate thereof: 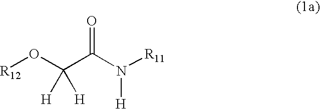
in which:
α) R₁₁ is selected from:
1) a C₁-C₁₀ alkyl radical optionally substituted with at least one substituent A₄,
2) a hydrocarbon-based ring C¹⁵ of 5 to 6 atoms optionally substituted with at least one substituent A₅;
β) R₁₂ is selected from:
1) a C₁-C₁₀ alkyl radical optionally substituted with at least one substituent A₄,
2) a hydrocarbon-based ring C¹⁶ of 5 to 6 atoms, optionally fused to at least one ring C¹⁷ of 5 to 6 atoms optionally containing at least one heteroatom to form a heterocycle Hy₁₇, these rings C¹⁶ and C¹⁷ optionally comprising a carbonyl function and/or being substituted with at least one substituent A₅;
γ) A₄ is selected from:
1) a ring C¹⁸ of 5 to 6 atoms optionally containing at least one heteroatom to form a heterocycle Hy₁₈, this ring or this heterocycle being optionally substituted with at least one substituent A₆ selected from among OR₁₄, CF₃, a halogen atom, and a C₁-C₁₀ alkyl radical;
2) one of the groups CF₃, CN, OR₁₃, SR₁₃, NR₁₃R′₁₃, NR₁₃C(═NR′₁₃)NR″₁₃R′₁₃, COR₁₃, COOR₁₃, CONR₁₃R′₁₃, NR₁₃COR′₁₃, NR₁₃CONR′₁₃R″₁₃, SO₂NR₁₃R′₁₃, NR₁₃SO₂R′₁₃, SO₂R₁₃, SiR₁₃R′₁₃R″₁₃, and SO₃H, in which R₁₃, R′₁₃, R″₁₃ and R₁₃′ independently represent a hydrogen atom or a C₁-C₁₀ alkyl radical;
δ) A₅ is selected from:
1) a halogen atom,
2) one of the groups CF₃, CN, OR₁₅, SR₁₅, NR₁₅R′₁₅, OCOR₁₅, COR₁₅, COOR₁₅, SO₂R₁₅, SiR₁₅R′₁₅R″₁₅, NO₂ and OCF₃, in which R₁₅, R′₁₅ and R″₁₅ independently represent a hydrogen atom or a C₁-C₁₀ alkyl radical optionally substituted with a phenyl radical,
3) a C₁-C₁₀ alkyl radical optionally substituted with a group OR₁₄,
4) a ring C¹⁹ of 5 to 6 atoms optionally containing at least one heteroatom to form a heterocycle Hy₁₉ and/or optionally comprising a carbonyl function;
ε) R₁₄ is selected from:
1) a hydrogen atom,
2) a C₁-C₁₀ alkyl radical;
η) Hy₁₇, Hy₁₈ and Hy₁₉ independently represent a heterocycle of 5 to 6 atoms optionally having from 1 to 4 heteroatoms selected from among N, O and S and combinations thereof and/or comprising a carbonyl function.

35. The topically applicable cosmetic/pharmaceutical composition as defined by claim 21, said at least one 2-oxyacetamide compound having one of the following formulae (II) to (V) or a salt and/or solvate thereof: 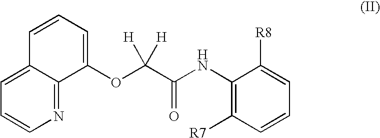
Formula (III): 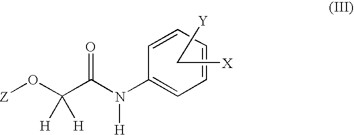
Formula (IV): 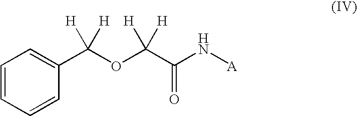
Formula (V): 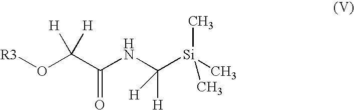
in which:
(i) R7 and R8 independently represent:
1) a hydrogen atom,
2) a halogen atom F or Cl,
3) one of the groups CF₃, CN, OR₄, SR₄, NHR₆, NR₆R′₆, OCOR₄, COR₄, CSR₄, COOR₄, SO₂R₄, SiR₄R′₄R″₄ and OCF₃, in which R₄, R′₄ and R″₄ independently represent a hydrogen atom or a C₁-C₂₀ alkyl radical optionally substituted with a phenyl radical and in which R₆ and R′₆ represent a C₁-C₂₀ alkyl radical optionally substituted with a phenyl radical,
4) a C₁-C₂₀ alkyl radical optionally substituted with a group OR₅ in which R₅ is a hydrogen atom or a C₁-C₂₀ alkyl radical,
5) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function;
(ii) Z is one of the following rings and heterocycles: 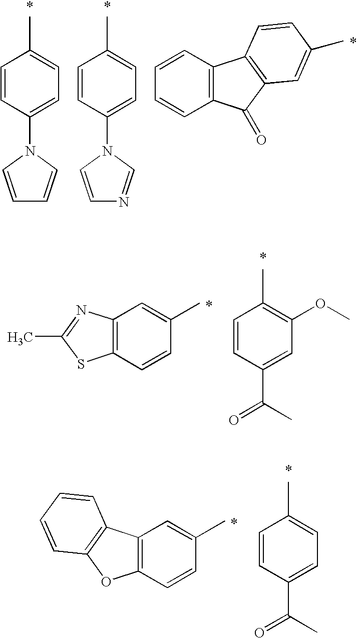
(iii) X and Y independently represent a hydrogen atomor A₂ or may form a fused ring C¹³ of 4 to 7 atoms, this ring C¹³ optionally comprising at least one heteroatom, being optionally substituted with at least one substituent A₂, and optionally fused to another ring C¹⁴;
(iv) A is one of the following three groups: 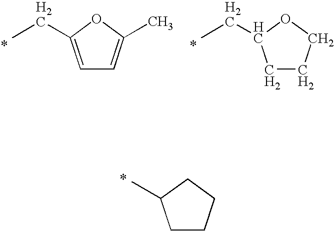
(v) R₃ is as defined for formula (I); with the proviso that when Z is the dibenzofuran heterocycle, X and Y independently represent:
1) a hydrogen atom,
2) a halogen atom,
3) one of the groups CF₃, CN, OR₉, SR₄, NR₄R′₄, OCOR₄, COR₄, CSR₄, COOR₄, SO₂R₄, SiR₄R′₄R″₄, NO₂ and OCF₃, in which R₄, R′₄ and R″₄ independently represent a hydrogen atom or a C₁-C₂₀ alkyl radical optionally substituted with a phenyl radical and in which R₉ is a hydrogen atom or a C₂-C₂₀ alkyl radical optionally substituted with a phenyl radical,
4) a C₂-C₂₀ alkyl radical optionally substituted with a group OR₅,
5) a ring C¹¹ of 4 to 7 atoms optionally containing at least one heteroatom to form a heterocycle Hy₁₁, and/or being optionally fused to a ring C¹² of 4 to 7 atoms, these rings C¹¹ and C¹² optionally comprising a carbonyl or thiocarbonyl function; or with the proviso that when Z is the ring: 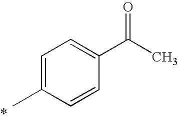
X and Y independently represent:
1) a hydrogen atom, in this case X is different than Y,
2) fluorine,
3) one of the groups OR₁₀, SR₄, NR₄R′₄, OCOR₄, COR₄, CSR₄, COOR₆, SiR₄R′₄R″₄ and OCF₃, in which R₄, R′₄ and R″₄ independently represent a hydrogen atom or a C₁-C₂₀ alkyl radical optionally substituted with a phenyl radical, in which R₆ is a C₁-C₂₀ alkyl radical optionally substituted with a phenyl radical and in which R₁₀ is a hydrogen atom or a benzyl radical or a C₃-C₂₀ alkyl radical optionally substituted with a phenyl radical,
4) a C₂-C₂₀ alkyl radical optionally substituted with a group OR₅,
5) with the proviso that X and Y may also form a 5-atom ring C¹³ optionally containing one or two oxygen atoms and being optionally fused to another ring C¹⁴.

36. The topically applicable cosmetic/pharmaceutical composition as defined by claim 21, said at least one 2-oxyacetamide compound having one of the following formulae or a salt and/or solvate thereof:

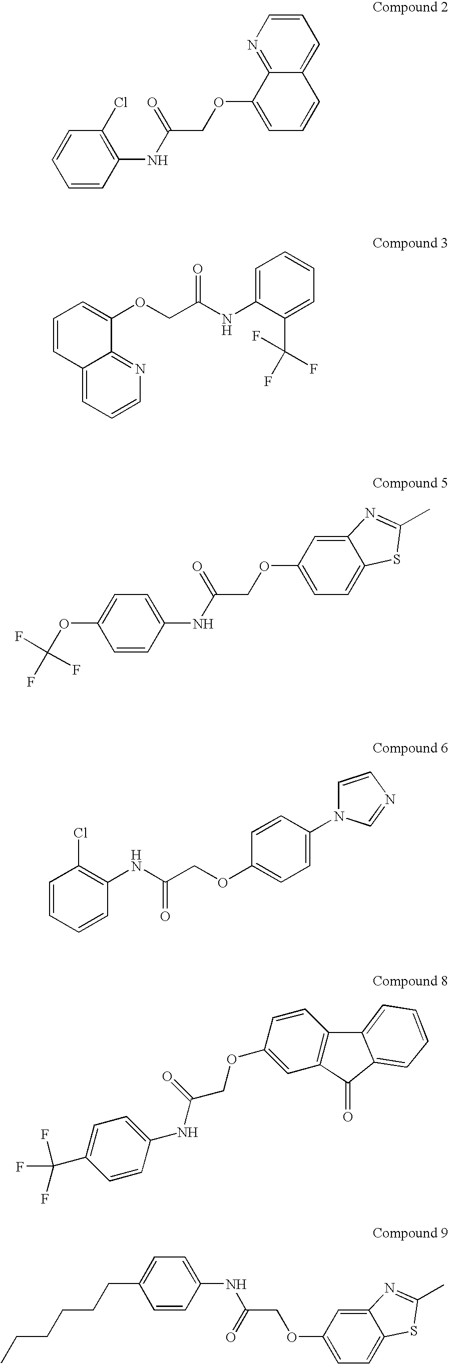

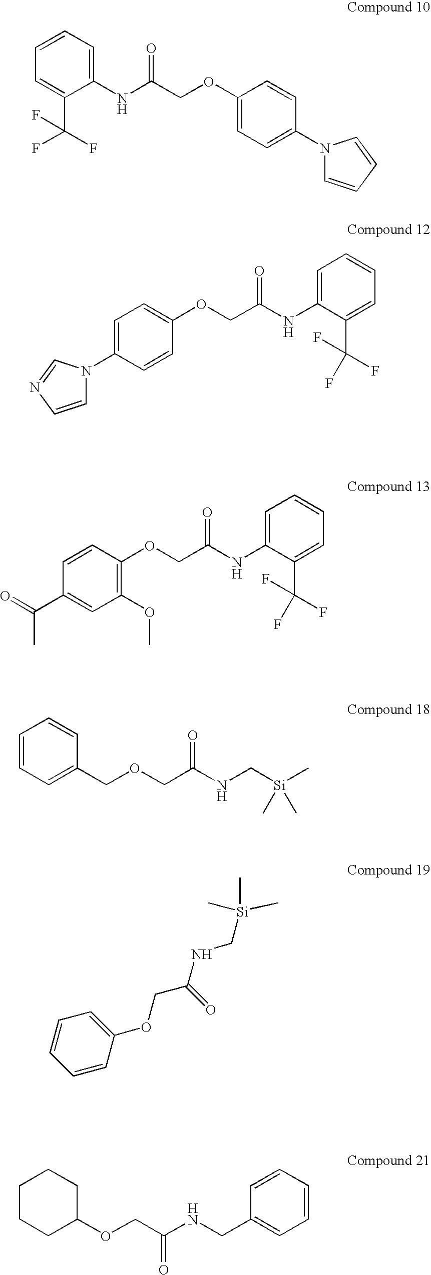

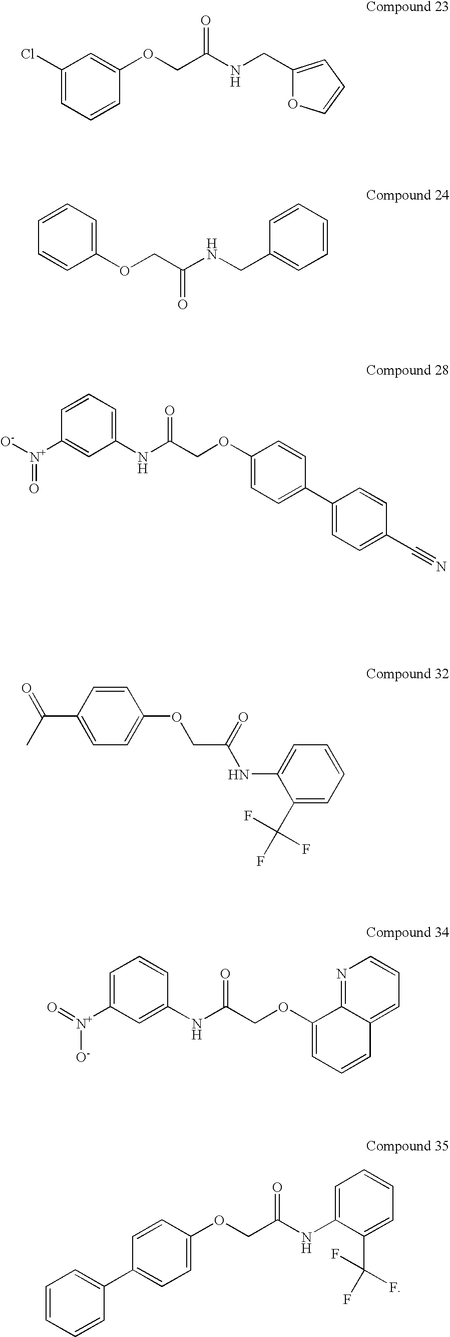

37. The topically applicable cosmetic/pharmaceutical composition as defined by claim 21, further comprising at least one other ingredient selected from the group consisting of solvents, aqueous-phase or oily-phase solvents, thickeners or gelling agents, dyes that are soluble in the medium of the composition, solid particles such as fillers or pigments, antioxidants, preservatives, fragrances, electrolytes, neutralizers, film-forming polymers, UV blockers, for instance sunscreens, cosmetic and pharmaceutical active agents with a beneficial effect on the skin or keratin fibers, other than the compounds of formula (I), and mixtures thereof.

38. The topically applicable cosmetic/pharmaceutical composition as defined by claim 21, further comprising at least one active agent selected from the group consisting of proteins and protein hydrolyzates, amino acids, polyols, urea, allantoin, sugars and sugar derivatives, water-soluble vitamins, plant extracts, hydroxy acids, retinol and derivatives thereof, tocopherol and derivatives thereof, essential fatty acids, ceramides, essential oils, salicylic acid derivatives, hydroxy acid esters and phospholipids, and mixtures thereof.

39. The topically applicable cosmetic/pharmaceutical composition as defined by claim 21, further comprising at least one additional active agent that promotes the growth and/or limits the loss of keratin fibers.

40. The topically applicable cosmetic/pharmaceutical composition as defined by claim 39, said at least one additional active agent being selected form the group consisting of [(9Z,12Z)octadeca-9,12-dienoyl]hexapyranose, lipoxygenase inhibitors, bradykinin inhibitors, prostaglandins and derivatives thereof, prostaglandin receptor agonists or antagonists, non-prostanoic prostaglandin analogs, vasodilators, anti-androgens, cyclosporins and analogs thereof, anti-microbial agents, anti-inflammatory agents, retinoids, benzalkonium chloride, benzethonium chloride, phenol, estradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharide or acylhexosaccharide acids, substituted aryl ethylenes, N-acylamino acids, flavonoids, ascomycin derivatives and analogs, histamine antagonists, saponins, proteoglycanase inhibitors, estrogen agonists and antagonists, pseudoterines, cytokines, growth factor promoters, IL-1 or IL-6 inhibitors, IL-10 promoters, TNF inhibitors, vitamins, benzophenones, hydantoin, retinoic acid, anti-pruriginous agents, anti-parasitic agents, anti-fungal agents, calcium antagonists, hormones, triterpenes, anti-androgenic agents, steroidal or non-steroidal 5-α-reductase inhibitors, potassium channel agonists and FP receptor antagonists, and mixtures thereof.

41. The topically applicable cosmetic/pharmaceutical composition as defined by claim 40, said at least one additional active agent being selected from the group consisting of aminexil, FP receptor agonists, prostaglandins and derivatives thereof, and vasodilators.

42. The topically applicable cosmetic/pharmaceutical composition as defined by claim 40, said at least one additional active agent being selected from the group consisting of aminexil, minoxidil, latanoprost, (5E)-7-{(1R,2R,3R,5S)-3,5-dihydroxy-2-[(3R)-3-hydroxy-5-phenylpentyl]cyclopentyl}hept-5-enoic acid, butaprost and travoprost.

43. A cosmetic regime or regimen for treating human keratin fibers and/or the skin from which said fibers emerge, in order to improve their condition and/or appearance, comprising topically applying onto said keratin fibers and/or the skin a cosmetic composition which comprises at least one compound of formula (I) as defined in claim 21 or a salt and/or solvate thereof, maintaining the composition in contact with the keratin fibers and/or the skin from which said fibers emerge, and optionally rinsing said fibers and/or said skin.

44. A cosmetic regime or regimen for treating the hair and/or the scalp, comprising topically applying onto the hair and/or the scalp a cosmetic composition which comprises a thus effective amount of at least one compound of formula (I) as defined in claim 21 or a salt and/or solvate thereof, maintaining the composition in contact with the hair and/or the scalp, and optionally rinsing it out.

45. A cosmetic regime or regimen for caring for and/or making up human eyelashes and/or eyelids, in order to improve their condition and/or appearance, comprising topically applying onto the eyelashes and/or the eyelids a mascara composition which comprises at least one compound of formula (I) as defined in claim 21 or a salt and/or solvate thereof and maintaining the composition in contact with the eyelashes and/or the eyelids.

46. A 2-oxyacetamide compound having one of the following formulae (II) to (V) or a salt and/or solvate thereof:
Formula (II): 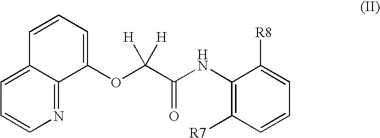
Formula (III): 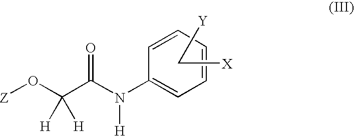
Formula (IV): 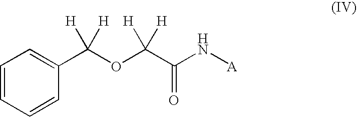
Formula (V): 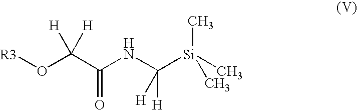
in which:
(i) R7 and R8 independently represent:
1) a hydrogen atom,
2) a halogen atom F or Cl,
3) one of the groups CF₃, CN, OR₄, SR₄, NHR₆, NR₆R′₆, OCOR₄, COR₄, CSR₄, COOR₄, SO₂R₄, SiR₄R′₄R″₄ and OCF₃, in which R₄, R′₄ and R″₄ independently represent a hydrogen atom or a linear or branched and especially saturated C₁-C₂₀ alkyl radical optionally substituted with a phenyl radical and in which R₆ and R′₆ represent a C₁-C₂₀ alkyl radical optionally substituted with a phenyl radical,
4) a C₁-C₂₀ alkyl radical optionally substituted with a group OR₅ in which R₅ is a hydrogen atom or a linear or branched and especially saturated C₁-C₂₀ alkyl radical,
5) a ring C¹¹ of 4 to 7 atoms optionally containing at least one heteroatom to form a heterocycle Hy₁₁, and/or being optionally fused to a saturated or unsaturated ring C¹² of 4 to 7 atoms, these rings C¹¹ and C¹² optionally comprising a carbonyl or thiocarbonyl function;
(ii) Z is one of the following rings and heterocycles: 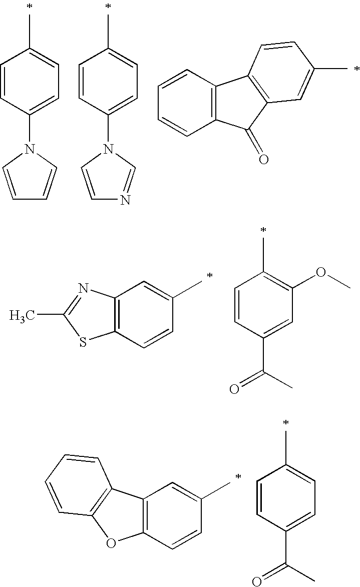
(iii) X and Y independently represent a hydrogen atom or A₂ as defined above (cf. formula (I)) or may form a fused, saturated or unsaturated ring C¹³ of 4 to 7 atoms, this ring optionally comprising at least one heteroatom selected from among N, O, S and combinations thereof and being optionally substituted with at least one substituent A₂ and optionally fused to another ring C¹⁴;
(iv) A is one of the following three groups: 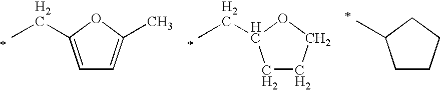
(v) R₃ is as defined in formula (I) with the proviso that when Z is the dibenzofuran heterocycle, X and Y independently represent:
1) a hydrogen atom,
2) a halogen atom,
3) one of the groups CF₃, CN, OR₄, SR₄, NHR₆, NR₆R′₆, OCOR₄, COR₄, CSR₄, COOR₄, SO₂R₄, SiR₄R′₄R″₄ and OCF₃, in which R₄, R′₄ and R″₄ independently represent a hydrogen atom or a C₁-C₂₀ alkyl radical optionally substituted with a phenyl radical and in which R₉ is a hydrogen atom or a C₂-C₂₀ alkyl radical optionally substituted with a phenyl radical,
4) a C₂-C₂₀ alkyl radical optionally substituted with a group OR₅,
5) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function; or with the proviso that when Z is the ring: 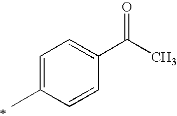
X and Y independently represent:
1) a hydrogen atom, in this case X is different than Y,
2) fluorine,
3) one of the groups OR₁₀, SR₄, NR₄R′₄, OCOR₄, COR₄, CSR₄, COOR₆, SiR₄R′₄R″₄ and OCF₃, in which R₄, R′₄ and R″₄ independently represent a hydrogen atom or a C₁-C₂₀ alkyl radical optionally substituted with a phenyl radical, in which R₆ is a C₁-C₂₀ alkyl radical optionally substituted with a phenyl radical and in which R₁₀ is a hydrogen atom or a benzyl radical or a C₃-C₂₀ alkyl radical optionally substituted with a phenyl radical,
4) a C₂-C₂₀ alkyl radical optionally substituted with a group OR₅,
5) X and Y may also form a 5-membered (or 5-atom) ring C¹³ optionally containing one or two oxygen atoms and being optionally fused to another ring C¹⁴.

47. A 2-oxyacetamide compound as defined by claim 46 having one of the following formulae or a salt and/or solvate thereof: [chemical structure]
